(19) United States
(12) Patent Application Publication
(10) Pub. No.: US 2026/0249010 A1
(43) Pub. Date: Aug. 27, 2026

(54) SEQUENTIAL DELIVERY SYSTEMS AND METHODS

(71) Applicant: Windgap Medical, Inc., Watertown, MA (US)

(72) Inventors: Andrew John Ryan, Milton, MA (US); Daniel Yasevac, Needham, MA (US); Jameson Woods, Watertown, MA (US); Phillip A. Soucy, Watertown, MA (US); Jeffrey Thomas Chagnon, Somerville, MA (US)

(21) Appl. No.: 19/649,985

(22) Filed: Apr. 16, 2026

Related U.S. Application Data
(63) Continuation of application No. PCT/US24/51795, filed on Oct. 17, 2024.
(60) Provisional application No. 63/590,958, filed on Oct. 17, 2023.
(60) Provisional application No. 63/622,886, filed on Jan. 19, 2024.

(51) Int. Cl. A61M 5/19; A61M 5/20; A61M 5/24; A61M 5/315; A61M 39/24

(52) U.S. Cl. CPC A61M 5/19; A61M 5/2033; A61M 5/2053; A61M 5/2455; A61M 5/31568; A61M 39/24; A61M 2039/248

(57) ABSTRACT

An automated, and/or semi-automated, sequential drug delivery injector system, where the energy provided to dispense medicament from a plurality of containers or cartridges can be iterated to deliver the medicament(s) in discrete steps, without mixing of the medicament components within the device. A pressurized gas source can drive the flow through a plurality of valves to sequentially dispense a dose, or discrete drugs, from each of the plurality of containers housed within the device. The delivery needle is in fluid communication with a flow path linking the adjacent drug containers when in the nominal state.

### CROSS REFERENCE TO RELATED APPLICATIONS

This application is a Continuation of International Application No. PCT/US24/51795, filed on Oct. 17, 2024, which claims the benefit of priority to U.S. Provisional No. 63/590,958 filed on Oct. 17, 2023, and U.S. Provisional No. 63/622,886, filed on Jan. 19, 2024; the entire content of each application is hereby incorporated by reference.

### BACKGROUND OF THE DISCLOSED SUBJECT MATTER

### Field of the Disclosed Subject Matter

The present invention relates generally to dual container devices for sequentially delivering medicament components (e.g. different medicament components, or multiple doses of a single medicament component). In addition to working well for large volumes of active pharmaceutical ingredient(s) including those of high viscosity requiring a large amount of force, or an API that requires a large amount of agitation for maximum efficacy. The devices disclosed herein can scale or be sized to accommodate standard drug cartridges, e.g. from 1 mL to 5 mL in volume each but cartridges less than 1 mL and larger than 5 mL are also contemplated.

### Description of Related Art

Dual container/cartridge injector/autoinjectors are known, typically for storing drug components separately until reconstitution or mixing at point of use, or co-administered separately from two containers. There are various benefits to therapeutics which may be preferred to be provided in a multi-chamber format. The drug may be less thermally stable, have a shorter shelf life, or have other issues being in its aqueous form. Solubilizing drugs in liquid agents, suspending dry particles in liquids, or combining liquid-liquid solutions or suspensions thereof may be required for similar reasons. In other cases, multiple liquid drugs that need to be co-administered may not suitable for storage in the same container due to stability, different requirements around pH, or molecule interaction issues that can impact efficacy of the drugs themselves.

In some cases, speed and ease-of-use may be critical for rescue applications where an emergency treatment needs to be delivered very quickly and with very few steps. Preparation can also require multiple steps that include changing out needles, or moving drug and diluent from one container to another manually. Moreover, if a second medicament, and or second dose of a common medicament is required, traditional devices can require a second injection operation and in some instances a separate needle assembly for administering.

As a result of these additional user-required step, users may experience: delays in treatment time, inadequate dosage amounts, or become generally dissatisfied with the experience of using the product. In other cases, drugs may be formulated in less ideal ways where users may be required to inject a higher dose volume, endure a less comfortable dosage form, a larger than desirable delivery needle, be exposed to additional solubilizing or stabilizing agents added to the formulation, or be required to make more frequent injections. There is significant motivation to create a device that can improve upon the sequential delivery of drugs which are otherwise cannot be co-formulated, difficult to solubilize, reconstitute, or suspend by re-combination alone.

The present application seeks to solve some of these identified problems as well as other problems that will become apparent to those skilled in the art.

### SUMMARY OF THE DISCLOSED SUBJECT MATTER

The purpose and advantages of the disclosed subject matter will be set forth in and apparent from the description that follows, as well as will be learned by practice of the disclosed subject matter. Additional advantages of the disclosed subject matter will be realized and attained by the methods and systems particularly pointed out in the written description and claims hereof, as well as from the appended drawings.

To achieve these and other advantages and in accordance with the purpose of the disclosed subject matter, as embodied and broadly described, the disclosed subject matter includes a drug delivery system comprising: a housing configured to hold a first container and a second container, wherein the first container contains a first medicament component and the second container contains a second medicament component; a first seal associated with the first container; a second seal associated with the second container; a fluid communication assembly having a fluidic channel between the first container and the second container, the fluid communication assembly configured to be displaced from a first position to a second position within the housing thereby opening, removing or otherwise piercing the first seal and second seal to provide a fluidic pathway between the first container and the second container; at least one actuator which can be a stored energy source configured to dispense the first and second medicament components; and a needle delivery assembly including a delivery needle having a first end and a second end, the first end of the delivery needle in fluid communication with the fluidic channel in the first position.

In some embodiments, the at least one stored energy source includes a spring engaged with a first plunger of the first container and a second spring engaged with a second plunger of the second container.

In some embodiments, the system further comprises a plate coupled to the first and second plungers, the plate restricting displacement of at least one of the first and second plungers.

In some embodiments, at least one of the first plunger and second plunger includes a structural feature configured to displace the plate, thereby permitting displacement of the other plunger.

In some embodiments, the at least one stored energy source includes at least one pressurized gas chamber, the at least one pressurized gas chamber in communication with the first container via a first pathway and the second container via a second pathway.

In some embodiments, the system further comprises a valve disposed between the at least one gas chamber and at least one of the first and/or second containers, the valve selectively opening flow of pressurized gas to one of the first and/or second containers.

In some embodiments, the system further comprises a third pathway in communication with the at least one pressurized gas chamber and at least one of the first pathway and second pathway.

In some embodiments, the at least one pressurized gas displaces a plunger in the first container a predetermined distance to open the second pathway to the second container.

In some embodiments, the at least one gas chamber includes a first gas chamber in fluid communication with the first container and a second gas chamber in fluid communication with the second container.

In some embodiments, the first gas chamber displaces a first plunger in the first container a predetermined distance to open the second gas chamber, thereby displacing a second plunger in the second container.

In some embodiments, the predetermined distance is detected by a sensor.

In accordance with another aspect of the disclosure, a drug delivery system is provided which comprises: a housing configured to hold a first container and a second container, wherein the first container contains a first medicament component and the second container contains a second medicament component; a first seal associated with the first container; a second seal associated with the second container; a fluid communication assembly having a fluidic channel between the first container and the second container, the fluid communication assembly configured to be displaced from a first position to a second position within the housing thereby opening, removing or otherwise piercing the first seal and second seal to provide a fluidic pathway between the first container and the second container; a pressurized gas chamber at least partially disposed in the housing and in fluid communication with the first container and the second container; an activation mechanism configured to open or otherwise pierce the pressurized gas chamber; at least one valve configured to release a portion of pressurized gas that facilitates the dispensing of the first and second medicaments components; and a needle delivery assembly configured to be in fluid communication with the first and second containers during a delivery phase.

In some embodiments, the pressurized gas flows through a first pressurized gas pathway to displace a plunger in the first container a predetermined distance, thereby opening a second pressurized gas pathway to the second container.

In some embodiments, pressurized gas remains within the first pressurized gas pathway while the pressurized gas flows to the second pressurized pathway.

In some embodiments, the first medicament and second medicament are dispensed sequentially and automatically.

In some embodiments, the pressurized gas chamber is disposed above the first container and a second container.

In accordance with another aspect of the disclosure, a drug delivery system is provided comprising: a housing configured to hold a first container and a second container, wherein the first container contains a first medicament component and the second container contains a second medicament component; a first seal associated with the first container; a second seal associated with the second container; a fluid communication assembly having a fluidic channel between the first container and the second container, the fluid communication assembly configured to be displaced from a first position to a second position within the housing thereby opening, removing or otherwise piercing the first seal and second seal to provide a fluidic pathway between the first container and the second container; a pressurized gas chamber at least partially disposed in the housing and in fluid communication with the first container and the second container; an activation mechanism configured to open or otherwise pierce the pressurized gas chamber; at least one valve configured to release a portion of pressurized gas that facilitates the dispensing of the first and second medicaments components; and a needle delivery assembly configured to be in fluid communication with the first and second containers during a delivery phase; and a valve disposed within the fluidic channel between the first container and the second container.

In some embodiments, the valve closes the fluidic channel to the second container while the medicament from the first container is dispensed.

In some embodiments, the valve is a ball valve, with the first medicament component displacing the ball valve a first direction to close the fluid channel of the second container.

In some embodiments, the second medicament component displaces the ball valve a second direction to close the fluid channel of the first container.

It is to be understood that both the foregoing general description and the following detailed description are exemplary and are intended to provide further explanation of the disclosed subject matter claimed.

The accompanying drawings, which are incorporated in and constitute part of this specification, are included to illustrate and provide a further understanding of the method and system of the disclosed subject matter. Together with the description, the drawings serve to explain the principles of the disclosed subject matter.

### BRIEF DESCRIPTION OF THE DRAWINGS

A detailed description of various aspects, features, and embodiments of the subject matter described herein is provided with reference to the accompanying drawings, which are briefly described below. The drawings are illustrative and are not necessarily drawn to scale, with some components and features being exaggerated for clarity. The drawings illustrate various aspects and features of the present subject matter and may illustrate one or more embodiment(s) or example(s) of the present subject matter in whole or in part.

FIGS. 1-2 are schematic representations of an exemplary Large Volume Dual Chamber (LVDC) Primary Drug Container (PDC) architecture and Dual Cartridge Holder (DCH), in accordance with the disclosed subject matter. It should be noted that reference to “Dual Cartridge Holder” and “Cartridge Holder” will be used interchangeably herein.

FIGS. 3-5 are a schematic representations of exemplary stored energy sources (e.g. springs) for driving plungers and dispensing medicament(s).

FIGS. 6-11 are a schematic representations of additional exemplary stored energy sources (e.g. gas cannisters) for driving plungers and dispensing medicament(s).

FIG. 12 is a schematic representation of exemplary actuators (e.g. electrical control) for driving plungers and dispensing medicament(s).

FIGS. 13-16 are a schematic representations of exemplary device activation features (e.g. needle shield (FIGS. 13-15), and button (FIG. 16).

FIG. 17 is a schematic representation of a Sequential Delivery Autoinjector (SDA), in accordance with another aspect of the disclosed subject matter.

FIG. 18 is a series of front views of the SDA of FIG. 17 depicting use states.

FIG. 19 is a schematic exploded view of the SDA of FIG. 17 with part identifiers.

FIG. 20 are cross sectional views of the SDA of FIG. 17 with part identifiers.

FIG. 21 are cross sectional views of the DCH.

FIG. 22 is a cross-sectional view of the SDA distal end in the nominal state.

FIG. 23 is a cross-sectional view illustrating a Safety Cap and Sterility Barrier being removed from SDA.

FIG. 24 is a schematic isometric view of an Internal Frame impulse snap arms array interacting with corresponding ramps on Housing.

FIG. 25 are schematic front views of Internal Frame translation rotating Spring-Release CAM.

FIG. 26 is a schematic illustration of dual-ramp gas activation system.

FIG. 27 is a schematic isometric view (with transparency) of Pneumatic Manifold.

FIG. 28 is a schematic cross-sectional view illustrating exposure and introduction of Delivery Needle into intended site.

FIG. 29 is a schematic cross-sectional view illustrating Needle Shield pushing the Delivery Hub in the delivery position.

FIG. 30 is a schematic cross-sectional view of the Pneumatic Manifold (pressurized volume shown in blue) and DCH, illustrating drug being delivered from the first Drug Cartridge via the Flow Channel.

FIG. 31 is a schematic cross-sectional view illustrating how the translation of the first Piston reveals the Pneumatic Pathway within the Pneumatic Manifold, rerouting the pressurized gas to overhead the second Piston.

FIG. 32 is a schematic cross-sectional view of the Pneumatic Manifold (pressurized volume shown in blue) and DCH, illustrating Drug being delivered from the second Drug Cartridge via the Flow Channel.

FIG. 33 is a schematic cross-sectional view illustrating how the translation of the second Piston reveals the Pneumatic Vent within the Pneumatic Manifold, releasing the gas and depressurizing the Pneumatic Manifold.

FIG. 34 is a schematic isometric view showing the compressed Needle Shield Return Spring pushing on the Internal Frame and, indirectly, the Needle Shield (not shown).

FIG. 35 is a schematic isometric view showing relative motion of Needle Shield and Delivery Hub, and how the Compliant Members fall into the Recesses to transition to the Lockout State.

FIG. 36 is a side view of another embodiment of a sequential drug delivery device.

FIG. 37 is an exploded part view of the device of FIG. 36.

FIG. 38 is a schematic cross-sectional view of device of FIG. 36 distal end in nominal state.

FIG. 39 is a schematic view of the device with the cap removed.

FIG. 40 is a schematic view of an impulse mechanism.

FIG. 41 is a schematic view the primary drug container activation steps.

FIG. 42 is a schematic view of gas activation.

FIG. 43 is a schematic cross-sectional view of a gas flow path and piston movement.

FIG. 44 is a schematic cross-sectional view of Mixing Prevention.

FIGS. 45-47 are schematic cross-sectional views of lockout features.

FIG. 48 are schematic views of the various device states of operation.

### DETAILED DESCRIPTION OF AN EXEMPLARY EMBODIMENT

Reference will now be made in detail to exemplary embodiments of the disclosed subject matter, an example of which is illustrated in the accompanying drawings. The method and corresponding steps of the disclosed subject matter will be described in conjunction with the detailed description of the system.

To provide clarity, the applicants would like to provide context around certain terms used throughout this description that is in addition to their ordinary meaning.

Distal or distal end primarily refers to the end of the injector system having the components and features to drive the plungers. In contrast, proximal or proximal end refers to the end of the device where the plungers are being driven into. For example, in all of the embodiments disclosed the delivery needle is disposed on the proximal end of the injector systems. Additionally, the distal end of the delivery needle is the end that is receiving the medicament components, whereas the proximal end of the delivery needle is injecting the medicament components into a recipient or otherwise releasing the medicament components.

For purposes of this application the term container can include any component that is configured to hold a volume. For example, a cartridge, pre-filled syringe, a vial and so forth would be considered a container. Containers can have attachment points, removable or pierceable seals associated with them and have medicament components stored therein.

As noted, there is a need to improve upon sequential delivery devices to allow for large volumes of drug formulations via a single device. The inventors, who created the embodiments herein, have provided solutions to at least this noted problem as well as other problems that will become apparent upon reading this description.

In many of the embodiments provided herein there is provided a fluid communication system, that includes a pair of cartridge access/piercing needles, a fluidic channel and a frame. This system can be positioned in the housing in a fixed manner, where other systems engage into it, or it can movable in a distal and/or proximal manner to engage with the containers as well as needle delivery system. Greater detail and examples of this fluid communication system can be found in U.S. published application US2022/0001112 A1, US2022/0379033, and/or US 2022/0001112, each of which is hereby incorporated by reference in its entirety.

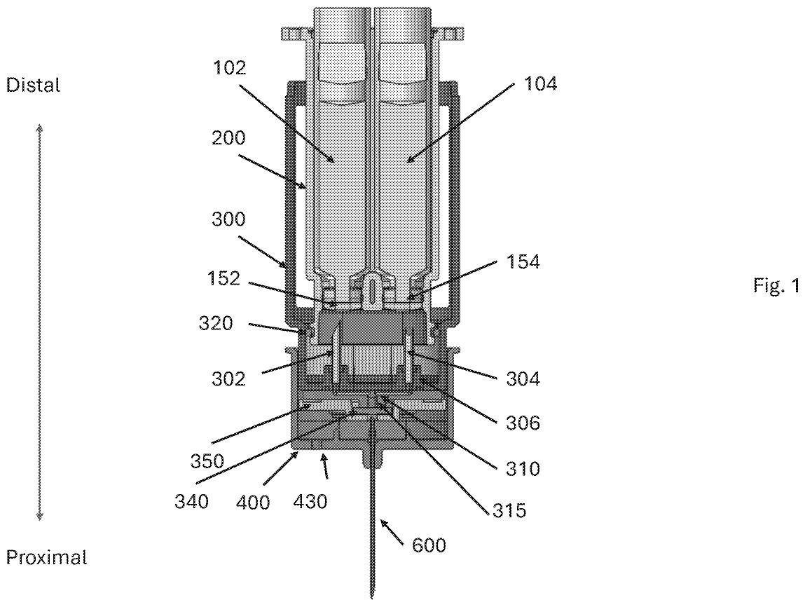

For purpose of explanation and illustration, and not limitation, exemplary embodiments of the system in accordance with the disclosed subject matter is shown in FIG. 1. Similar reference numerals (differentiated by the leading numeral) may be provided among the various views and Figures presented herein to denote functionally corresponding, but not necessarily identical structures.

The methods and systems presented herein may be used for large volume dual chamber (LVDC) primary drug container (PDC) which is used to facilitate storage, and delivery of a pharmaceutical ingredients. Two medicament components are held in separate drug cartridges 102, 104 within the device. It should be noted that reference to drug “cartridge” and drug “container” will be used interchangeably herein. In an exemplary embodiment, the PDC scales to accommodate standard drug cartridges from about, but not limited to, 1 mL to about 10 mL in volume, each. It is also contemplated in another embodiment (not shown) that the cartridges could be prefilled syringes.

The drug cartridges 102, 104 are held in the cartridge holder 200 to prevent them from moving during storage or use. The cartridges can be held via “interference-fit” or friction with adjacent structures/surfaces of the housing, and/or via mating engagement (e.g. mechanical interlock such as male/female complimentary surface features) to retain a fixed orientation with respect to the cartridge holder.

The cartridge holder 200 fits within the hub 300 such that the two components can be displaced (e.g. slide or translate vertically) relative to each other, but the walls of the hub 300 help maintain a specific orientation of the cartridge holder. For example, the upwardly extending walls of the hub 300 circumscribe at least a portion of the cartridge holder 200, thereby orienting the two components to have aligned central axes. In some embodiments, the hub 300 contains at least one (e.g. two equidistantly spaced) cartridge piercing needles affixed 302, 304 (e.g. adhesively attached, insert molded, or integrally formed) to the base of the component. Each cartridge piercing needle 302, 304 can be positioned below a central longitudinal axis of the drug cartridges 102, 104. Corresponding through holes concentric to the cartridge access/piercing needles 302, 304 are included in the bottom of the hub, such that anything that flows through the needles can flow through the hub 300 as well. Thus, these components form a fluid communication assembly of the cartridges 102, 104. In the embodiment with the prefilled syringe, alternative to the cartridge-based design, the septa 152, 154 would swap positions with the cartridge access/piercing needles 302, 304. That is, the needles 302, 304 would be directly staked into the drug cartridges 102, 104 (which can be made of glass or plastic) with the seals 152, 154 affixed and attached to the inlets of the channel.

Additional examples of cartridges 102,104, cartridge holder 200 and hub 300 are provided in International Patent Application No. PCT/US24/23643, the entire contents of which are hereby incorporated by reference.

In some embodiments, the cartridge access/piercing needles 302, 304 can be seated within upwardly extending needle receptacles 306 in the base of the missing hub, sized with an inner diameter sufficient to receive the outer diameter of the needles 302, 304 therein.

The medicament/drug delivery flow channel 310 contains a groove that connects the cartridge access/piercing needles 302, 304 in the hub 300. In operation the medicament/drug from each container 102, 104 travels though this channel, albeit at separate times so as to avoid mixing of the medicaments within the channel. To the extent there is any residual medicament within the flow channel 310 from the dispensing of the first medicament, due to the high viscosity of and laminar flow of the medicaments, the second medicament does not mix with said residual first medicament within the flow channel 310. This feature is applicable to all embodiments disclosed herein. To ensure that no fluid escapes from this groove of the flow channel 310, there is a second concentric groove that surrounds the central groove. This groove contains an O-Ring 320, or other sealing surface, that is compressed between the flow channel 310 and hub 300 creating a seal. In another embodiment the O-Ring could be a two-shot molded elastomer, molded directly into the cartridge holder 200 or hub 300.

Additionally, a through hole 315 is included in fluid communication with and disposed below the flow channel 310 which separates the flow channel 310 from the delivery needle 600 until the septum 340 is pierced by the delivery needle 600 allowing for fluid to exit out of the channel 310 (and downwardly into the delivery needle). In the exemplary embodiment shown, the through hole is located at the center of the device, equidistantly spaced between the two needles 302, 304, and vertically aligned with the delivery needle 600. At the end of the through hole is a septum 340 (which can be formed of a resilient elastomeric member) that is compressed against the flow channel 310 by the septum cap 350. The septum seals the through hole of the flow channel 310 (until being pierced or opened by the delivery needle 600, as described below).

The hub 300 (which can be referred to as a “release” hub, since the medicaments from each container 102, 104 are initially released within the flow channel 310 of this hub 300) along with flow channel 310, and septum cap 350 fit within the delivery hub 400. Similar to the hub 300 and cartridge holder 200, The delivery hub's upwardly extending walls help to guide the other components such that they can slide relative to each other with a specific orientation. In the exemplary embodiment the delivery hub 400 has upwardly extending sidewall that circumscribes at least a portion of the hub 300 received therein; and the hub 300 in turn receives the cartridge holder 200 therein (which contains the drug cartridges 102, 104), as described above. Thus, the device can be configured with a nesting arrangement, in cascading order, of: the drug cartridges, cartridge holder, hub and delivery hub.

Additionally, the delivery hub 400 has the delivery needle 600 affixed (e.g. glued, insert molded, or affixed in some other fashion) into its base that is used for delivery of the medicament components to its intended target. The delivery needle 600 can be located at the center of the delivery hub and extend both upwardly into the interior of the delivery hub 400, and downwardly beyond the lower boss on the bottom surface of the delivery hub 400. In another embodiment, the delivery needle is not located at the center of the delivery hub but offset by some amount.

### Sterility Features

The device disclosed herein contains many features that are specially used for sterility purposes. When stored, the primary drug container prevents ingress of particles and bacteria, or other bioburden or endotoxin, from reaching critical interfaces that could introduce such bacteria, bioburden, or endotoxin to the patient.

The first main area to ensure sterility is the hub compartment 300 created by the void in space between the bottom of the cartridge holder 200 and the inner surfaces of the hub 300. Seals are created to ensure that no particulate reaches the needles, or tops of the drug cartridges. The cartridges 102, 104 are press fit into the cartridge holder 200 to create a radial seal that no particulate can bypass.

In some embodiments, a seal is established between the cartridge holder 200 and hub 300. For purpose of illustration and not limitation, in an exemplary embodiment an O-Ring groove 319, with O-Ring 320 disposed therein, along the outer wall of the cartridge holder 200 creates a seal between the cartridge holder 200 and the hub 300. It should be noted that any reference to an O-Ring, of any kind, could be two-shot molded into a another part and may not be an isolated O-Ring, but simply denote a sealing surface for the purposes of preventing foreign particulate matter, bioburden, or endotoxin from crossing the interface.

The second compartment is the delivery hub compartment created by the hub 300 and the inner walls of the delivery hub 400. The delivery needle 600 is affixed (e.g. glued) into the delivery hub which prevents particulate from bypassing along the exterior surface of the needle.

The delivery hub 400 has a hole in its lower surface establishing the delivery hub compartment vent 430. This Vent is initially covered by a cover or film (e.g. Tyvec, or other foil) that could be inserted (e.g. ultrasonically welded) to the plastic to create a seal. The intent of the lower surface of the delivery hub is that a safety cap fits over the needle 600 and press fit around the lower boss that the delivery needle 600 protrudes from. This creates the final seal to enclose the delivery hub compartment 400 and delivery needle 600 and ensure all remain sterile prior to use.

### Device States of Operation

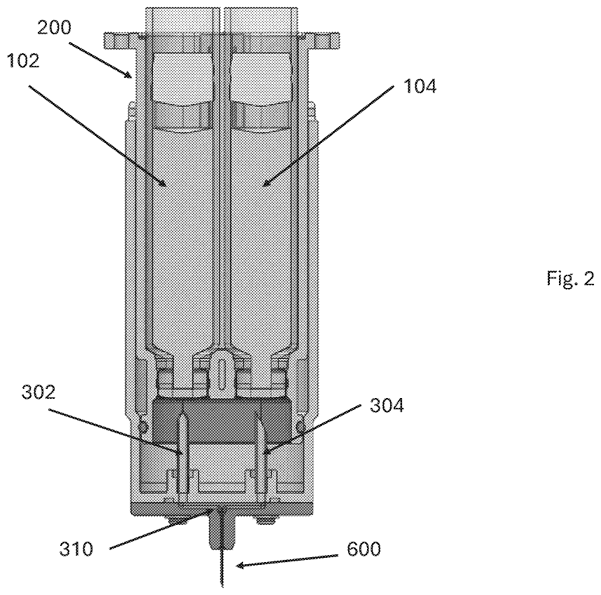

The device disclosed herein has a plurality (e.g. four) different device states throughout its operation life cycle: Nominal, Activated, Delivery of first medicament, and Delivery of second medicament. In the nominal state, the components are all assembled together as described above in connection with FIGS. 1-2, and the different compartment are all sealed and sterile.

When the hub 300 moves vertically up relative to the cartridge holder 200, the needles 302, 304 pierce the standard drug cartridges container closure septum (152, 154), and a fluid pathway is opened up between the two cartridges 102, 104 via the flow channel 310. The septum 340 below the flow channel 310 prevents any fluid from being released, while also providing an access point for the non-patient end of the delivery needle. The septum cap 350 ensures the septum 340 remains under compression to prevent leaking. The delivery hub 400 sits around the hub 300 and the other components it is fixated to and holds the delivery needle 600. When the delivery hub 400 moves vertically up relative to the rest of the assembly, the non-patient, proximal, end of the delivery needle 600 pierces the septum 340 and a fluid pathway is formed between the flow channel 310 and the patient.

The LVDC PDC provides the opportunity to complete sequential delivery of the two medicaments stored separately in each cartridge. Note, although reference is made to “two” medicaments this can include two discrete/different medicaments with differing formulations and active ingredients (as well as diluents or other component). Additionally, two doses (of equivalent or differing volume) of the same medicament can be provided in the two cartridges 102, 104. Moreover, additional medicament containers (i.e. greater than the two adjacent cartridges 102, 104 shown) can be incorporated into the housing so that the device can automatically and sequentially deliver any desired amount/number of medicaments to a patient.

This is beneficial when wanting to package different medicaments together for delivery to the patient, while maintaining only a single delivery needle. The PDC also allows for the simultaneous delivery of a plurality of standard cartridges of the same medicament, therefore increasing the total deliverable volume within one device. If desired, the PDC/DCH could also be used to simultaneously inject both drug cartridges at the same time of any similar or dissimilar medications stored in each drug cartridge.

In accordance with an aspect of the disclosure, and as shown in the exemplary embodiment shown in FIG. 2, the delivery hub 400 can be formed with a complimentary shape such that the upper portion of the delivery hub 400 forms a channel 310 geometry wherein a slot allows fluid access to both drug cartridges 102, 104. Here, the non-patient, proximal, end of the delivery needle 600 is already in the fluid flow path 310—that is, there is no need to pierce a septum 340, as shown in FIG. 1. The user, or device, does not need to impart any force or displace any components for the delivery needle 600 to be placed in fluid communication with the channel 310, as this is the default position of the needle 600. In this exemplary embodiment, once the hub 400 moves vertically and the needles 302,304 pierce the drug cartridges 102,104, delivery can be achieved. It should be noted that reference to “mixing” with respect to channel 310 does not mean that medicament from one cartridge mixes, or transfers, to blend with the medicament in the adjacent cartridge; but rather that each medicament will, in discrete sequences of delivery, travel through the same physical channel 310.

### Power Sources

There are many different power sources that could facilitate the sequential delivery of the plurality of medicaments stored in separate cartridges 102, 104. For example, the devices disclosed herein can use a stored energy source (which can include springs, pressurized gas cannisters, mechanical and/or electrical powered actuators). Reference to “actuators” and “stored energy sources” can be used interchangeably throughout this disclosure. In some embodiments, spring-based mechanisms can be employed (e.g. compression springs, extension springs, constant force springs, torsion springs, and clock springs, etc.). There can be separate springs that are each dedicated for driving the motion of the separate plungers in each individual cartridge 102,104. Alternatively, a single spring can be employed which is linked to a mechanism (e.g. plate) that controls the motion of both plungers.

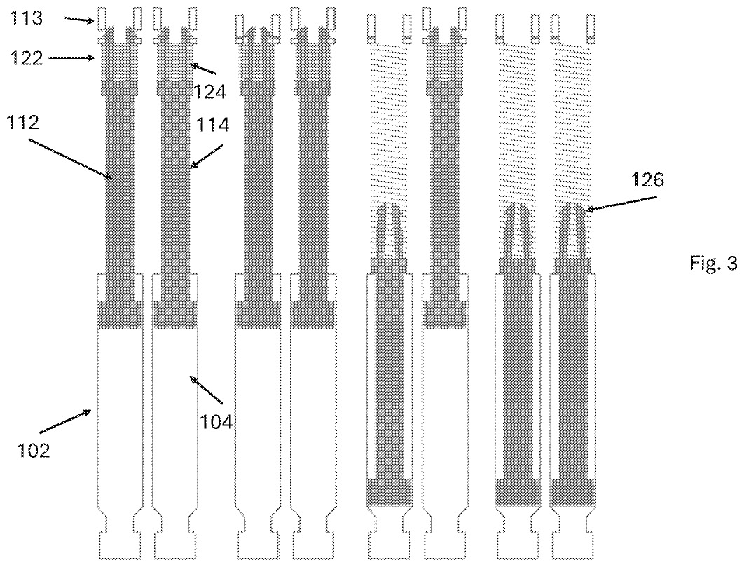

In the exemplary embodiment shown in FIG. 3, the two plunger rods 112, 114 are each driven by individual (e.g. constant force) springs 122, 124. The proximal end of the spring 122, 124 can abut against a flange of the medicament cartridge or cartridge holder 200, and the distal end of the spring engaged with the plunger rod to drive the rod distally into the medicament container (with the spring remaining external to the medicament container to avoid contamination of the medicament, and/or degradation of the spring). The plunger rod can include tabs 126 on the proximal end that locking engage the cartridge holder 200 when in the stored state, but can deflect inwardly to release the plunger to advance under the spring force to dispense the medicament from the containers.

The plunger rod 112 can include latches at the proximal end which releasably engage the actuator 113. As the actuator is pressed downward (due to the user pressing the device against the target area of the skin and compressing the device), the latches (which can be formed as inclined ramps) are squeezed together to disengage the actuator sidewalls 113, at which point the spring 122 is free to release its force and drive the plunger rod 112 downward to dispense the medicament within the container 102.

As shown in the third stage of FIG. 3, these release tabs 126 can operate independently such that the first plunger 112 may be released while the release tabs of the second plunger remain engaged/locked. This allows for the dispensing of each medicament container to be a discrete operation, while also permitting sequential dispensing of the medicament(s).

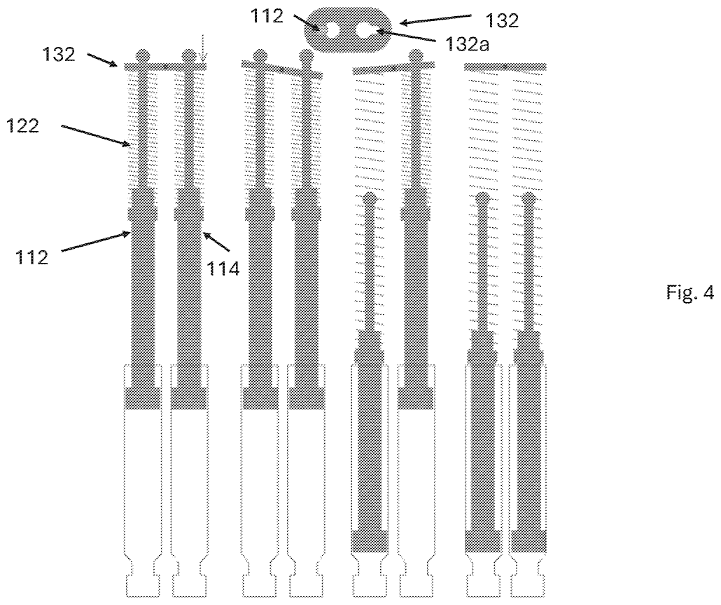

In the exemplary embodiment shown in FIG. 4, a mechanical linkage can couple the two plunger rods. For example, a plate 132 with apertures for receiving the distal ends of the plunger rods 112, 114. In the initial (i.e. stored) condition, the plungers are engaged/locked on the plate 132, preventing them from moving. The plate 132 has notches (132a) that the plungers 112, 114 can move into to begin delivery. For example, the plungers 112, 114 can be formed with a bulbous distal end with a greater diameter than the notch 132a portion of the plate 132 so that the plunger is restricted from longitudinal movement. To commence dispensing, the proximal end of the plunger is displaced laterally though the notch 132a to the portion of the notch with an equivalent (or greater) opening than the thickness/diameter of the bulbous plunger head so that the spring force displaces the plunger longitudinally (as shown in the third stag of FIG. 4).

Additionally or alternatively, the plate 132 can be displaced to actuate and release the plunger rods 112, 114. As shown in the second and third stages of FIG. 4, the plate 132 can be tilted, or depressed on one side, to align the larger opening of notch 132 with the plunger rod retention feature (e.g. bulbous head, but other geometries are well within the scope of the present disclosure) thereby releasing the spring force to displace the plunger rod through the medicament container and dispense the medicament. Once the first plunger rod 112 has reached the end of travel, a structural feature (e.g. protruding ramp) slides the locking plate 132 horizontally. Next, the second plunger rod 114, that was resting on top of the plate 132 during operation/travel of the first plunger rod 112, is released and the medicament within the second container 114 is delivered.

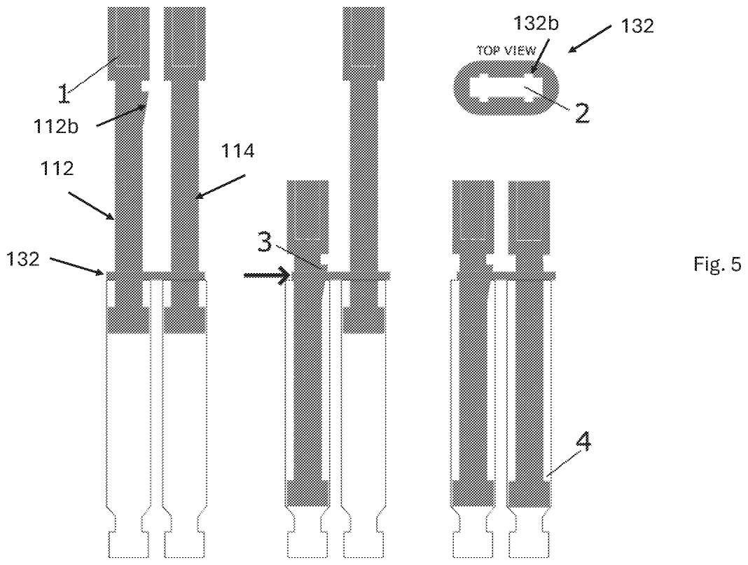

Another exemplary embodiment of a spring power source for driving sequential drug delivery is shown in FIG. 5. The springs can be contained within the plunger rods (as shown by Step 1) and the plate 132 can have notches (e.g. rectangular grooves) (shown as Step 2) with a greater size (or shape) opening than the remainder of the plate. Additionally, the plate 132 can be disposed near the distal end of the plungers 112, 114.

As shown in Step 1 of FIG. 5, the first plunger rod 112 is released to dispense medicament. The first plunger rod 112 can include a triggering structural feature 112b (e.g. rib or protrusion that tapers or projects outwardly near the proximal end of the plunger rod) to displace (e.g. laterally) the plate 132 and release the second plunger rod 114 for dispensing the medicament in the second container. That is, the operation of the second plunger 114 is prohibited until the first plunger rod 112 is displaced distally a sufficient distance so that the triggering geometric feature 112b engages the plate 132 to move the plate (e.g. laterally) to align the notch 132b with the second plunger rod, thereby releasing the second plunger rod for displacement (via spring force). These structural features ensure sequential delivery of the dose/drug within each cartridge, and inhibit or prohibit mixing of the dose/drug in situ. Displacement (e.g. lateral) of the plate 132 aligns the notch 132b with the second plunger rod 114 (shown as Step 3) to permit passage of the second plunger rod 114 through the plate 132 thereby driving dispensing of the medicament (shown as Step 4).

### Gas Power Source

In some embodiments of the present disclosure, the power source is gas power contained within a pressurized gas chamber or cannister (e.g. cylinder). For each of the embodiments disclosed herein, the gas chamber/cannister used can include a dual phase gas (e.g. one which is stored as a liquid and converts to a gas upon exiting the chamber/cannister and expanding to a larger volume), or a single phase gas (e.g. nitrogen). A mechanism (e.g. pin) is used to pierce the gas chamber and as the pressurized gas expands it powers the motion of the plungers in the drug cartridges. To do so effectively though, the pressure is controlled, e.g., via a regulator to lower the pressure, using an expansion volume to lower the pressure, using components that can operate at the high pressures of the cylinders, and/or limiting the flow rate into the cartridges to prevent a rapid increase in pressure.

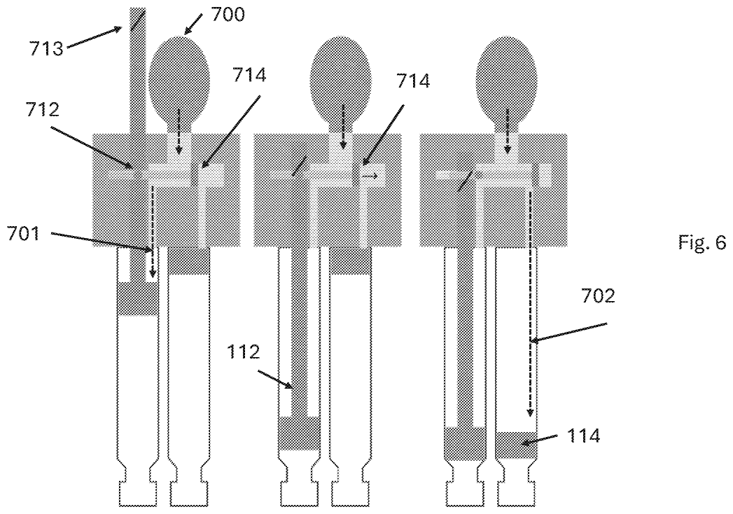

In the exemplary embodiment shown in FIG. 6, a compressed gas chamber 700 is included at the top of the plungers 112, 114, and a valve 712 operates to open a first air pathway 701 which delivers the first dose via plunger 112 while the second air pathway 702 to the second plunger 114 is blocked via barrier/seal 714 (as shown on the left side of FIG. 6). As shown, the first plunger rod 712 can extend upwardly beyond the cartridge 102 (and the gas manifold as well). Once the first plunger 112 is displaced a sufficient distance (e.g. to the end of the cartridge 102) a lever 713 on the proximal end of the first plunger rod 112 can activate, or push towards the right in the exemplary embodiment shown, to toggle or displace the seal 714 to open the pathway 702 to the second container 104. The pressurized gas continues to be released from the gas chamber 700 which gradually increases pressure in the manifold containing the valve 712, to provide pressurized gas to both the first container 102 via pathway 701, and due to displacement of the seal 714 (laterally to the right in the middle image of FIG. 6) to open the second pathway 702, the pressurized gas can now also be delivered to the second container 104 via the second pathway 702.

Once the second pathway 702 is opened, the pressurized gas in the manifold drives the second piston/plunger 114 downward to dispense the medicament in the second container. In some embodiments, the first fluid pathway 701 can be closed while pressurized gas displaces the seal 714 to open second fluid pathway 702 and drive the plunger to dispense medicament in the second container.

Additionally, as shown in FIG. 6, the first container can include a plunger rod 712 wherein the pressurized gas acts on the plunger rod to drive the rod (and piston disposed at an end thereof) to dispense medicament from the first container 102; while the pressurized gas acts directly on the piston in the second container 104. In other words, there is no plunger rod present in the second container 104, only the piston. In each of the embodiments disclosed herein which employ pressurized gas to dispense the medicament(s), the gas can act on the plunger rod, and/or directly on the piston or plunger head itself (in the event the plunger rod is not present in that particular container).

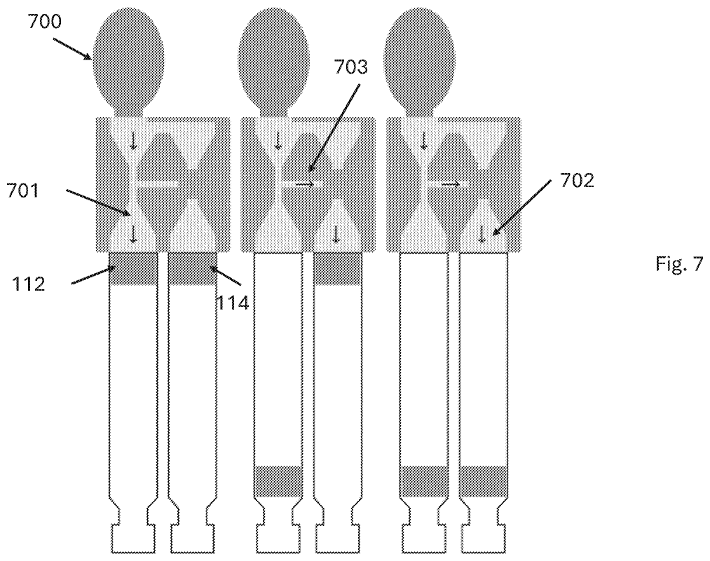

In the exemplary embodiment shown in FIG. 7, the gas chamber 700 delivers a pressurized gas to the first container 102 via the first fluid pathway 701, while the second fluid pathway is closed. The pressure continues to increase after the first plunger 112 has dispensed the medicament, to increase the pressure within a third pathway 703 to eventually open a valve (as shown in the middle of FIG. 7) to permit air to enter the second fluid pathway 702, to thereby drive the second plunger and dispense the second medicament. The third pathway 703 can oriented perpendicularly and extend between the midpoints of the first and second pathways 701, 702.

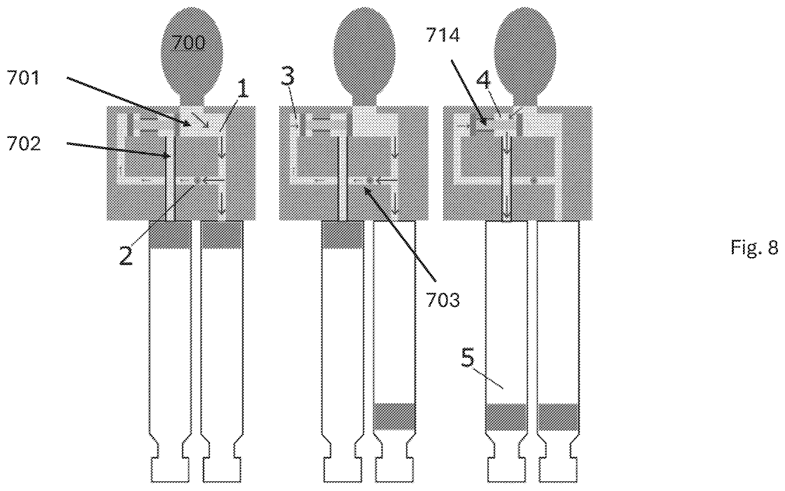

In the exemplary embodiment shown in FIG. 8, the gas cannister 700 delivers a pressurized gas to the first container via the first fluid pathway 701, while the second fluid pathway 702 is closed (as shown at Step 1 of FIG. 8). A third pathway 703, disposed between the first two pathways 701, 702, drives a pilot valve so that air flow is restricted to gradually increase pressure in this line (as shown at Step 2 of FIG. 8). As the pressure increases in the third pathway 703, the force applied against the shuttle piston 714 increases until it is displaces the the shuttle piston 714 (to the right, as shown at Step 4 of FIG. 8). Once shuttle piston is displaced (to the right in the depicted embodiment) the second pathway 702 is opened and pressurized gas then displaces the second plunger to dispense the second medicament (as shown at Step 5 of FIG. 8).

The gas pressure can be utilized in a variety of different ways to inject the medicament. The gas pressure can be directly put into the proximal end of the cartridges to push on the plunger. Additionally, or alternatively, the gas pressure can drive a piston or other mechanical mechanism that drives the plungers down. Additionally, or alternatively, the gas cylinders themselves could be used as pistons such that as pressure is released behind them the cylinder pushes down on the plunger or piston to drive the motion of the plunger.

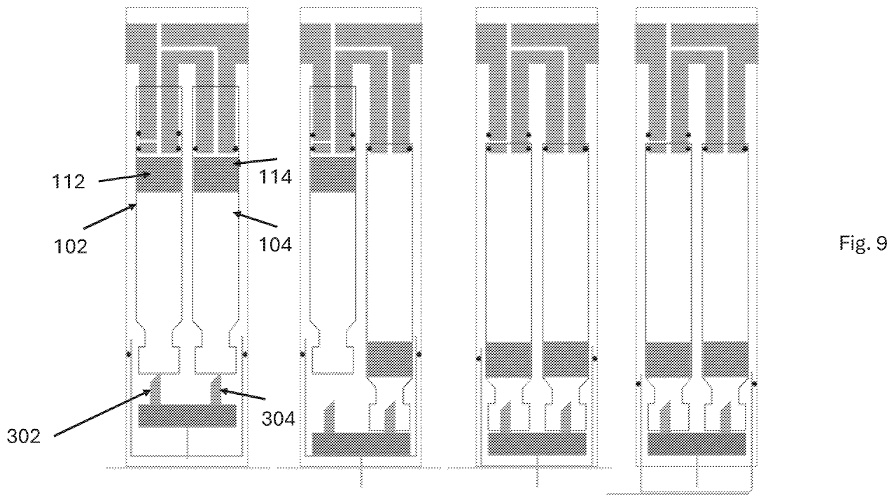

In the exemplary embodiment shown in FIG. 9, a first container 104 and first plunger 114 are driven downward to pierce the seal of container 104 on needle 304. Thereafter, the second container 102 and second plunger 112 are driven downward to pierce the seal of container 102 on needle 302.

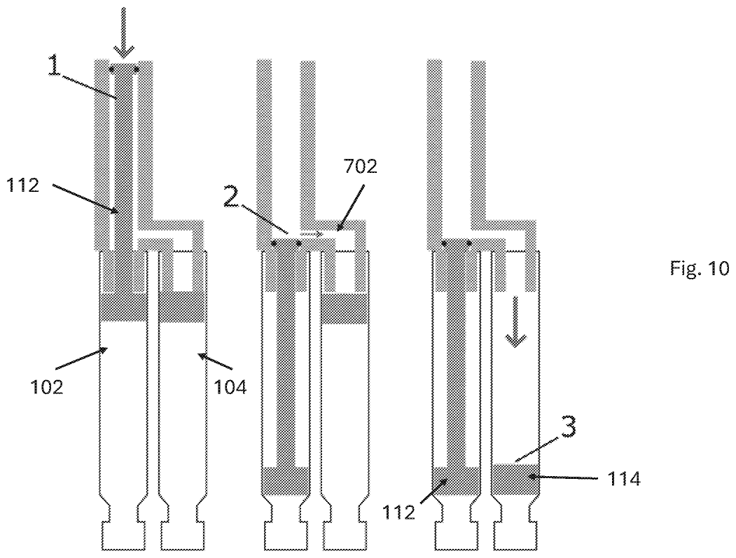

In the exemplary embodiment shown in FIG. 10, a first plunger rod 112 is driven down via pressurized air to deliver the first dose (shown as Step 1). Once the plunger rod 112 has reached the bottom of travel, a secondary air pathway 712 is revealed (or opened) to the second cartridge 104 (shown as Step 2). The pressurized gas then travels through the, now accessible, pathway 712 to displace the second plunger 114 and dispense the second medicament (shown as Step 3). As noted herein, some embodiments employ an elongated plunger rod attached to a plunger piston head, and some embodiments employ only the plunger piston/head (i.e. no elongated plunger rod). In the exemplary embodiment shown in FIG. 10, the first cartridge employs a plunger rod coupled to the plunger head, with the elongated rod serving as a seal blocking flow of pressurized gas to the second cartridge-until the plunger rod has been displaced a sufficient distance to remove the seal/blockage and permit pressurized gas flow to reach the second cartridge. The second cartridge does not require an elongated plunger rod, and hence only utilizes the plunger 114. This can save space in the housing and overall form factor of the device as there is no need to provide a cavity or void to receive the retracted end of the plunger rod (for the second cartridge).

Similar to springs, the device could use either a single gas cylinder to drive the motion of both plungers, or use multiple gas cylinders that individually control the separate drug cartridges. For multiple gas cylinders, the device could separately activate the two cylinders in sequence to control the sequential delivery of the medicament. Alternatively, after the first cylinder is pierced the motion of the plunger could allow for a mechanical mechanism to activate the second cylinder and in turn the delivery of the second cartridge.

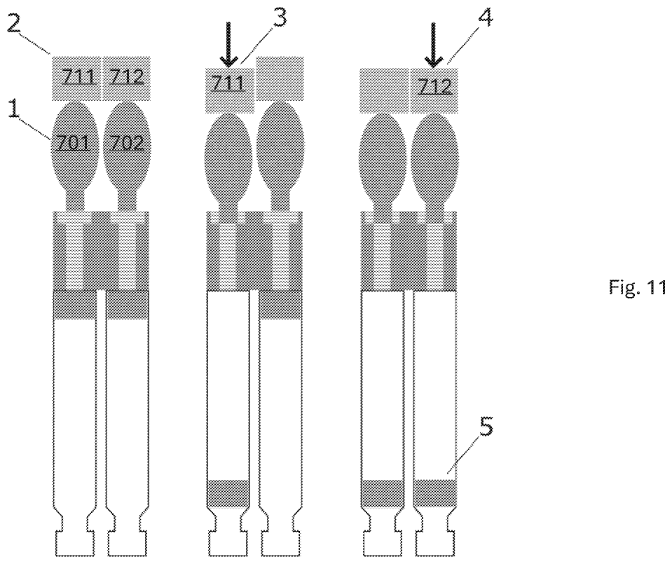

In the exemplary embodiment shown in FIG. 11, the sequential drug delivery device is powered by two separate gas cartridges 701, 702. Each gas cartridge is activated individually using a button 711, 712, so that when the first button 711 is pressed, the first gas cartridge 701 is activated which delivers the first dose (shown as Step 3 in FIG. 11). When the second button 702 is pressed, the second gas cartridge is activated, and the second dose is delivered (shown as Step 4 in FIG. 11).

In some embodiments, when a single gas cylinder is used, a mechanism (e.g. valve(s)) is employed to direct the power of the pressurized gas from one cylinder to the next. Some valving options allow for automatic delivery of the second dose such as a pilot valve or electronically controlled valve that senses the end of the first delivery. Other valve options require a user input such as a traditional stem valve. Alternatively, pneumatic pathways could also be configured such that after the first delivery is completed, a new pneumatic pathway is revealed to allow for the delivery of the second dose.

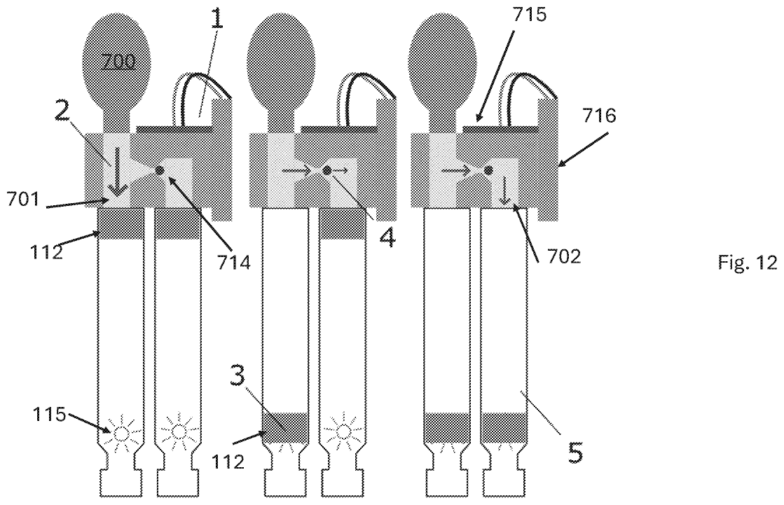

In the exemplary embodiment shown in FIG. 12, medicament delivery is controlled by an electronic controller (e.g. solenoid valve 714 driven by a control board 715, and powered by a battery 716). The valve is powered/commanded to open (shown as Step 1) to open the first compressed air pathway 701 (shown as Step 2) which delivers the first dose while the second air pathway is blocked. Once the plunger 112 reaches the bottom of travel, the plunger 112 passes in front of a light source 115 which triggers a light sensor (shown as Step 3). The trigger activates the solenoid which reveals the second air pathway 702 (shown as Step 4). The pressurized gas then drives the second plunger to dispense the medicament in the second container in a sequential manner.

The exemplary embodiment shown depicts pressurized gas cannisters and solenoid valves, but it will be apparent to artisans of ordinary skill that other power sources can be employed, including electric motors, chemical reactions, magnets, electromagnets, or user generated power.

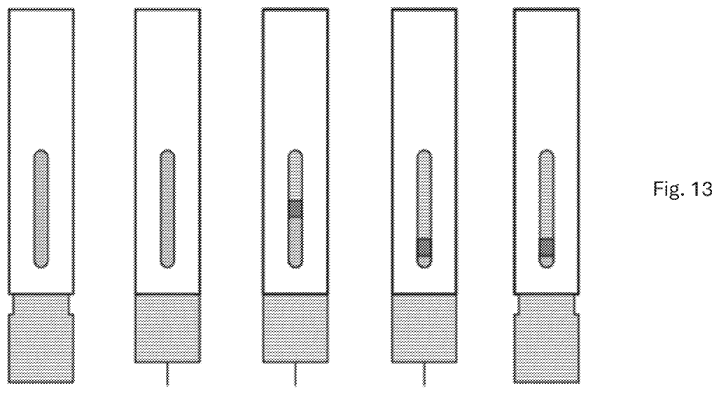

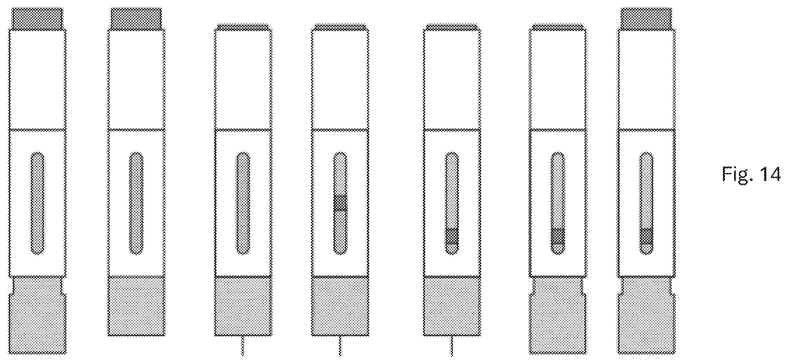

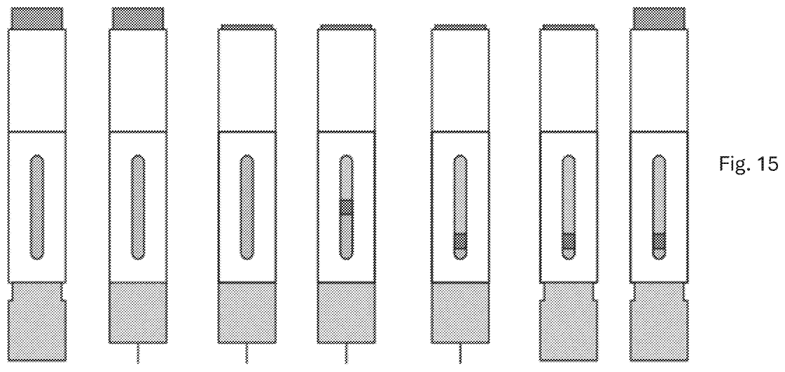

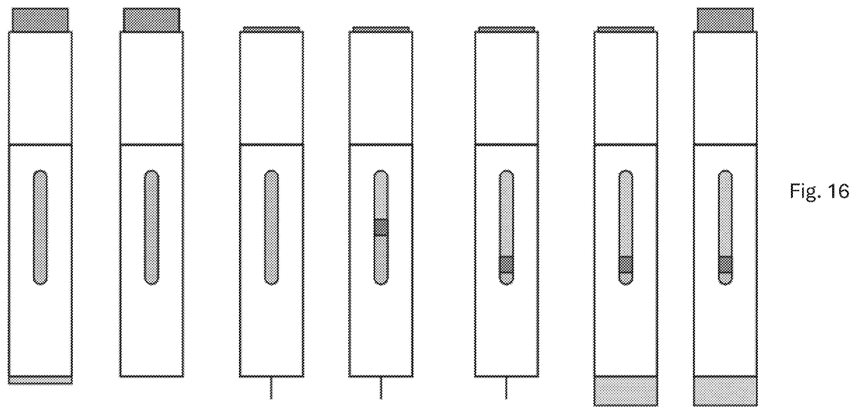

Depending on the power source and desired user input, multiple options for device activation are available. The device can be activated (medicament cartridges punctured and drug delivery initiated) in several different ways. For example, the device can utilize a needle shield that once depressed, inserts the needle into the patient, activates the hub and performs the delivery (as shown in FIG. 13). Similarly, a needle shield could be used to activate the hub, while a separate button inserts the needle into the patient and delivers the drug (as shown in FIG. 14). The needle shield could be used to insert the needle into the patient and activate the hub with a separate button used only for the delivery (as shown in FIG. 15). Additionally or alternatively, a button can activate the hub, insert the needle into the patient, and perform the delivery with a protective needle shield activating at the end of the therapy (as shown in FIG. 16). All devices will have an additional safety cap that needs to be removed before the device can be used. There is an option to have the removal of this cap activate the hub while a separate button inserts the needle into the patient and performs the delivery.

While the exemplary embodiments describe a full depression of each plunger over the entire range of motion to the distal end of the containers, only a partial depression of either (or both) plungers is within the scope of the present disclosure. Thus, any desired amount of medicament from either container can be administered.

In accordance with an aspect of the disclosure, the automatic sequential delivery autoinjector is built around the large volume dual chamber primary drug container configuration to allow a user to deliver large volumes of high viscosity drugs. The user controls the activation and point of delivery, but the delivery force is controlled by the device. This design removes as many user steps as possible to ensure that delivery would not be affected by the user.

### Additional Embodiment Of Sequential Drug Delivery Device

In accordance with another aspect of the disclosure, a sequential delivery device (SDA) is provided that allows a user to automatically, or semi-automatically, deliver two doses of drug sequentially from a single device. The user controls the activation and point of delivery, but the delivery force is controlled by the device. Advantageously, this reduces user the number of user steps for delivery and ensures that the delivery timing and performance is not be affected by the user.

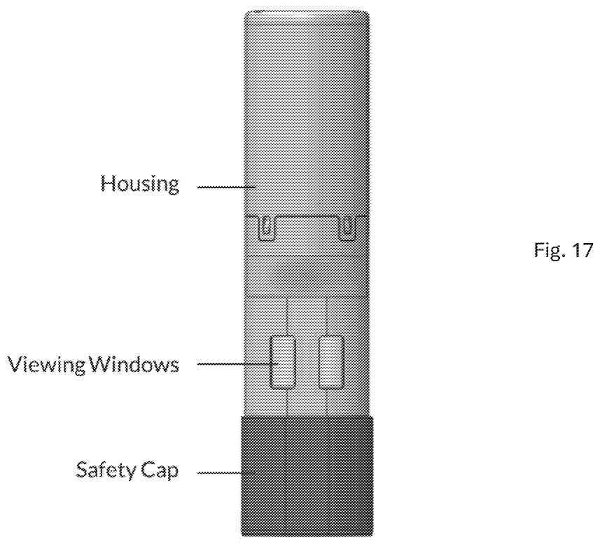

As depicted in the exemplary embodiment of FIG. 17, the device contains a Housing that acts as the main body for the user to hold during device use. The primary touch points for this device are the Safety Cap that is removed just prior to delivery and the Needle Shield (not shown in Figure as concealed under the Safety Cap) which is exposed, when the Safety Cap is removed, and acts as the activation trigger. The user can also obtain information about the device's state by observing a Viewing Window located on the side of the Housing, which allows the user to see into the drug cartridges and evaluate the medicament components.

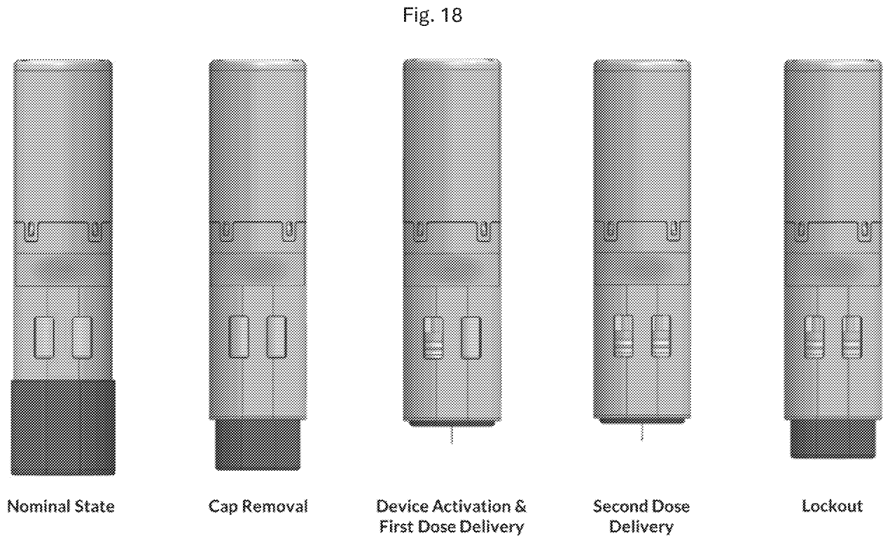

As shown in FIG. 18, when the user removes the device from its packaging, the device is in its Nominal State. There is stored energy within the device, isolated from the drug(s), and the Safety Cap is installed on the device. To activate the device, the user removes the Safety Cap to expose the Needle Shield. The user places the Needle Shield up against the injection site on the patient and presses the device into the patient. This activates the Pneumatic System of the device and causes the Delivery Needle to pierce the skin and open the Delivery Fluid Pathway. Once the Delivery is complete the User can pull the device away from the Patient and the Needle Shield will extend and lockout.

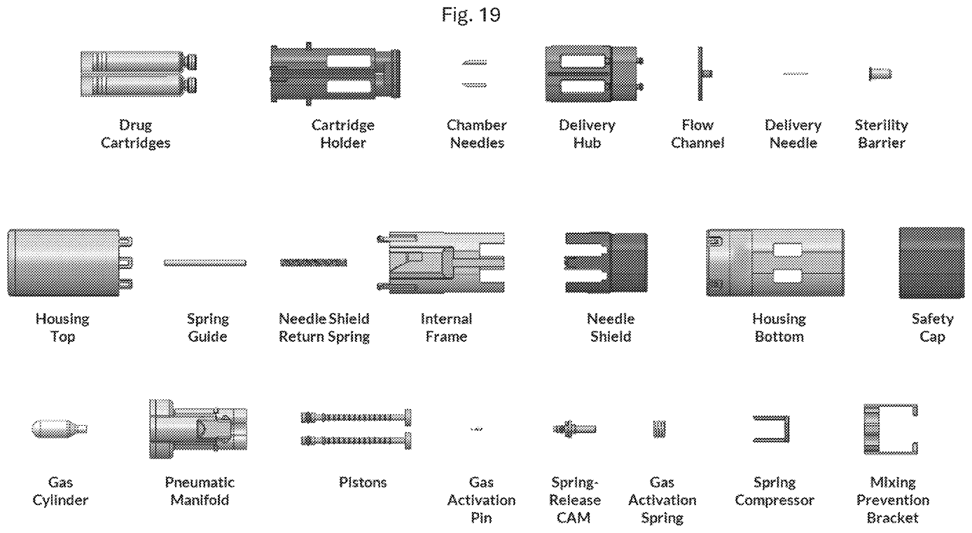

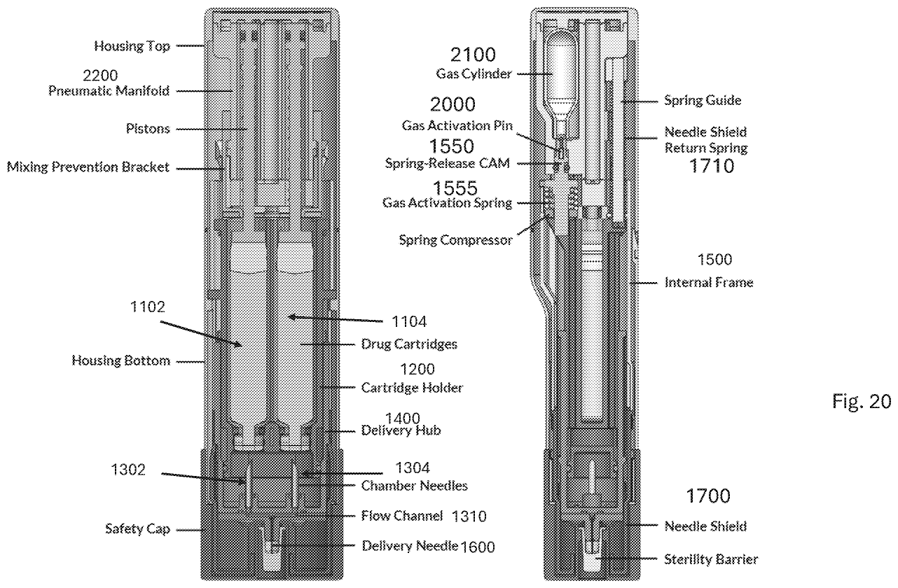

In the exemplary embodiment of the SDA, there are three main subassemblies.
1. The Dual Cartridge Holder (DCH) is comprised of two Drug Cartridges—including a Stopper or Plunger, drug (which may be the same or different formulations and volumes), Septum, and Septum Crimps—a Cartridge Holder, two cartridge piercing needles (labeled “Chamber Needles” in FIG. 19), a Delivery Hub, a Flow Channel, a Delivery Needle, and a Sterility Barrier.
2. The Power System is comprised of a Gas Cylinder, a Pneumatic Manifold, two Pistons, a Gas Activation Pin, a Spring-Release CAM, a Gas Activation Spring, a Spring Compressor, and a Mixing Prevention Bracket.
3. Finally, the Shroud is comprised of a Housing Top, a Spring Guide, a Needle Shield Return Spring, an Internal Frame, a Needle Shield, a Housing Bottom, a Safety Cap.
   FIGS. 19 and 20 illustrate the appearance of these parts and their relative location within the assembly.

With reference to FIG. 20, the Drug Cartridges 1102, 1104 are held in the Cartridge Holder 1200 to prevent them from moving during storage or use. The Cartridge Holder 1200 fits within the Delivery Hub 1400 such that the two can slide relative to each other, but the walls of the Delivery Hub help maintain a specific orientation of the cartridge holder. The Delivery Hub contains two cartridge piercing needles 1302, 1304 glued into the base of the component. There are through-holes concentric to the cartridge piercing needles 1302, 1304 such that anything that flows through the needles can flow through the Delivery Hub as well. The bottom face of the Delivery Hub contains four posts that help align and secure both the Flow Channel 1310 and the Drug Cartridges 1102, 1104.

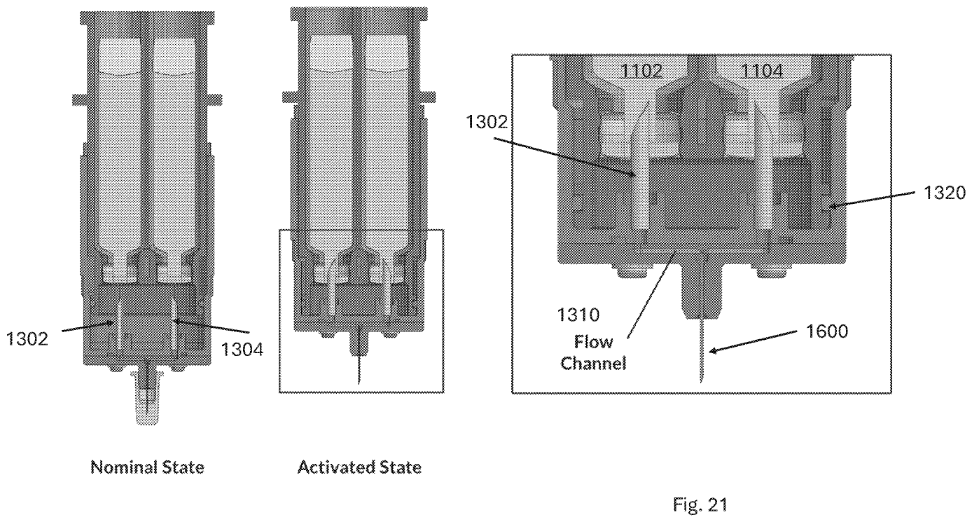

The Flow Channel 1310 contains a groove that connects the two cartridge piercing needles 1302, 1304 in the Delivery Hub 1400. This groove allows fluid and air to flow between the two cartridge piercing needles 1302, 1304 when an external force is applied to the Drug Cartridge Stopper. To ensure that no fluid escapes from this groove, a second groove that surrounds the central groove can be included. This radially outer groove contains an O-Ring 1320 that is compressed between the Flow Channel 1310 and Delivery Hub 1400 creating a seal (as best seen in FIG. 21).

Additionally, a through-hole in the Flow Channel 1310 to allow for fluid to not only pass between the two cartridge piercing needles 102, 1304, but out of the channel as well. At the end of the through-hole is a Delivery Needle 1600 glued into the Flow Channel 1310 that is used for delivering the drug to its intended target.

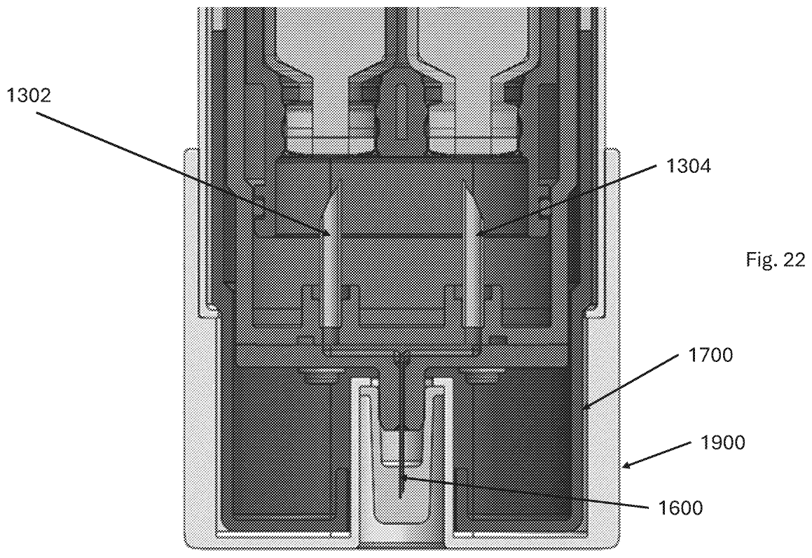

FIG. 22 depicts the sequential delivery autoinjector (SDA) in the Nominal State, where the Safety Cap 1900 captures a Sterility Barrier, which maintains Delivery Needle 1600 sterility during storage. Users can inspect the drug(s) before using the device via Viewing Windows (as shown in FIG. 17). The Safety Cap 1900 also prevents inadvertent activation of the device during inspection and preparation by preventing users from interacting with the Needle Shield 1700.

### Activation

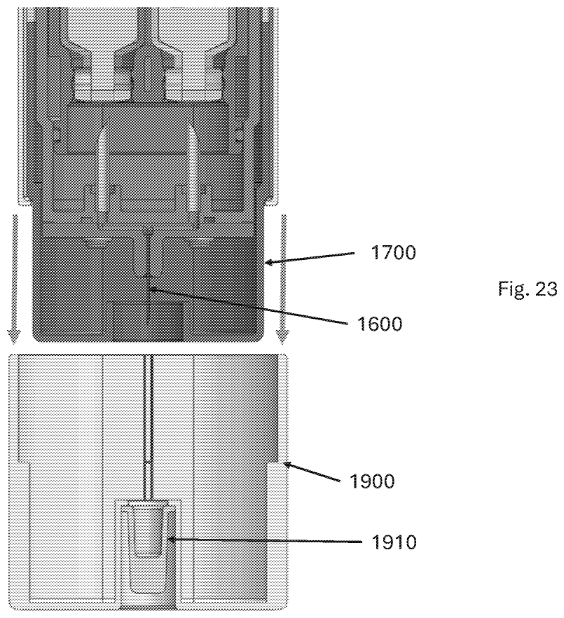

As shown in FIG. 23, when the user is ready to perform the injection, they firmly grasp the Safety Cap 1900 and pull axially relative to the SDA Housing. As the Safety Cap 1900 is removed, the Sterility Barrier 1910 is also removed from the Delivery Needle 1600. Once the Safety Cap 1900 is removed, the SDA is ready to deliver. The Needle Shield 1700 still protects user from accidental pricks by the Delivery Needle 1600 while they position the SDA.

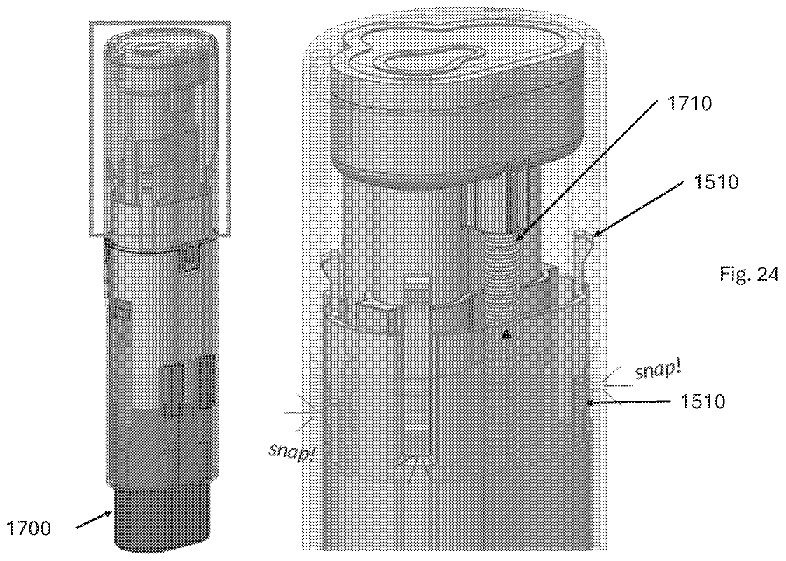

Next, the user aligns the SDA with the intended target on the patient and begins firmly pressing the Needle Shield 1700 into the intended target. As the user presses, the Internal Frame is pushed by Needle Shield 1700, resisting the user's force. This is due to interference between an array of impulse snap arms 1510 on the Internal Frame 1500 and corresponding ramps within the device Housing, as shown in FIG. 24. Once sufficient force is generated to overcome the deflection force of all the impulse snaps arms 1510, the impulse snap arms deflect around the ramps and the Internal Frame 1500, as well as the Needle Shield 1700, begin translating within the Housing. The SDA has now been activated, and the next series of device states are achieved by utilizing the residual downward force provided by the user. The exact timing of each mechanism may change relative to the other steps.

As the Internal Frame 1500 translates, it compresses the Needle Shield Return Spring 1710. The Needle Shield Return Spring 1710 will later provide the force required to re-extend the Needle Shield 1700 after delivery is completed.

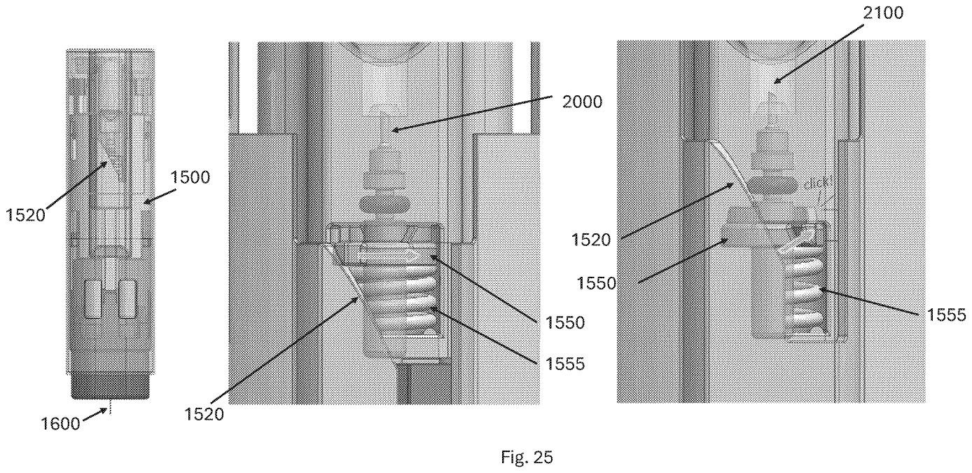

Additionally, as shown in FIG. 25, as the Internal Frame 1500 translates, a ramp surface 1520 on the Internal Frame contacts a Spring-Release CAM 1550, causing the Spring-Release CAM to rotate. In the stored state, the Spring-Release CAM 1550 is held in compression between the Pneumatic Manifold and the Gas Activation Spring 1555. After sufficient rotation, raised features on the Spring-Release CAM will align with recesses within the Pneumatic Manifold, allowing the Gas Activation Spring to push the Spring-Release CAM into the Pneumatic Manifold. Ramps on the Spring-Release CAM complete the rotation after aligning with the recesses, preventing obstruction of further Internal Frame translation. As the Spring-Release CAM is pushed further into the Pneumatics Manifold, the Gas Activation Pin 2000 pierces the Gas Cylinder 2100, releasing the gas stored inside the Gas Cylinder and activating the Pneumatic System. A seal on the Spring-Release CAM prevents the gas from escaping the Pneumatic Manifold.

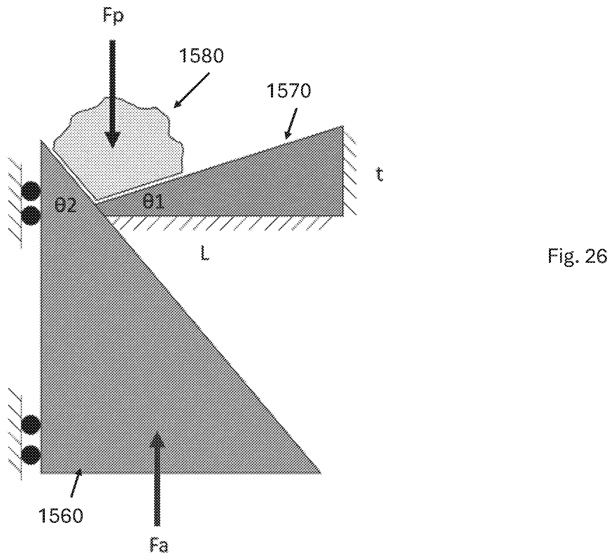

In another embodiment of gas activation, a dual ramp system, as illustrated in FIG. 26, can be implemented. In this embodiment, the large ramp 1560 represents the translation of the Internal Frame 1500, the smaller ramp 1570 represents a fixed ramp on the Pneumatic Manifold, and mass 1580 represents the Spring-Release CAM. There is no spring in this embodiment. As the Internal frame 1500 translates, the Spring-Release CAM is double-wedged between the two ramps 1560, 1570, causing a vertical translation. Alternatively, the snap release mechanism described in further detail below cam also be employed.

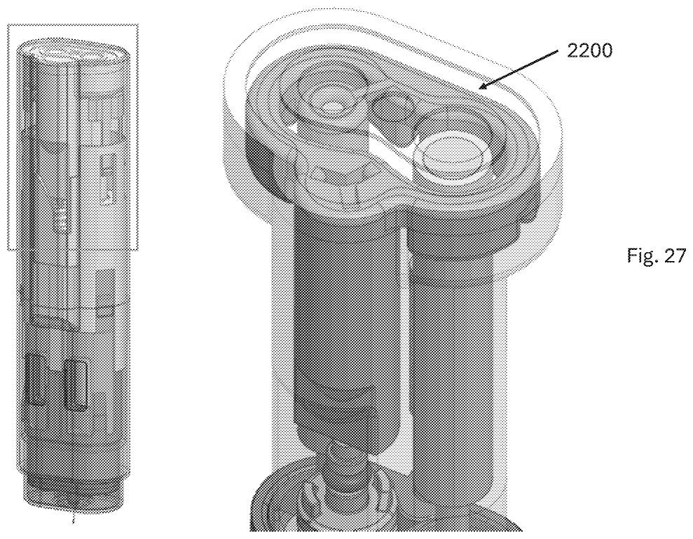

Once the gas is released, it rapidly fills the expansion volume in the Pneumatic Manifold (see FIG. 27). This reduces the gas storage pressure down to the operating pressure. Simultaneously, the Pneumatic Manifold routes the gas overhead of the first Piston for driving downward to dispense the medicament within the first medicament container 1102.

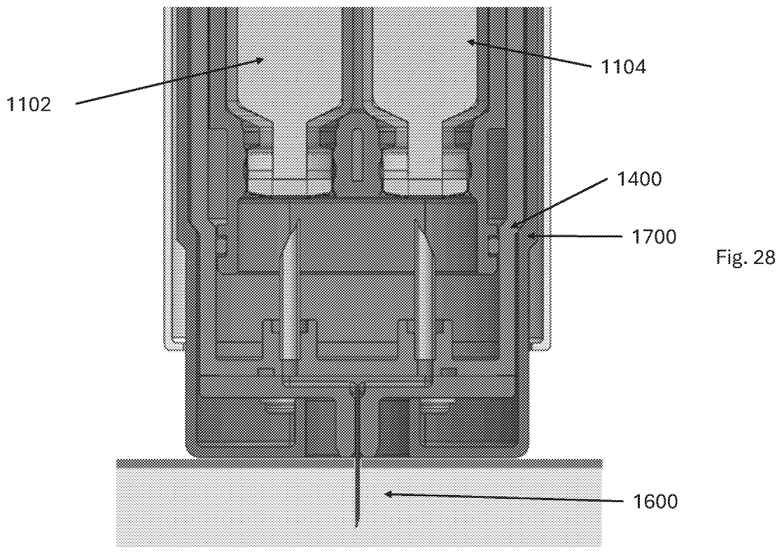

Before, after, or simultaneously with gas activation, the Needle Shield 1700 and Internal Frame are still translating. As the Needle Shield 1700 translates, the Delivery Needle 1600 is exposed and introduced into the intended location, as shown in FIG. 28. The exposure and introduction of the Delivery Needle 1600 continues until the Needle Shield 1700 contacts the DCH Delivery Hub 1400, corresponding to the full exposure and introduction of the of the Delivery Needle 1600. Even though the Drug Cartridges 1101, 1102 within DCH may be partially pressurized at this time, the DCH has not been activated yet, so no drug will be delivered.

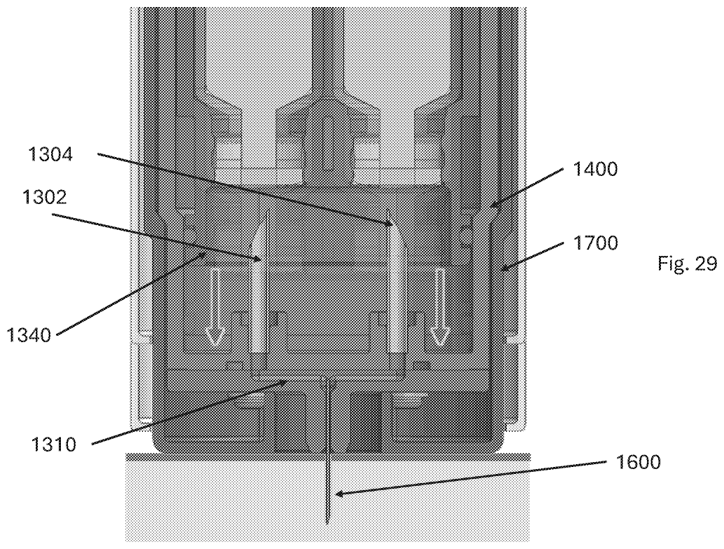

Finally, during the final length of Needle Shield 1700 translation, the DCH Delivery Hub 1400 is pushed into the delivery position, as shown in FIG. 29. In doing so, the cartridge piercing needles 1302, 1304 pierce the Septa 1340, exposing the drug to the Flow Channel 1310 as described above. This marks the transition of the device from the Activation State to the Delivery State.

### Sequential Delivery

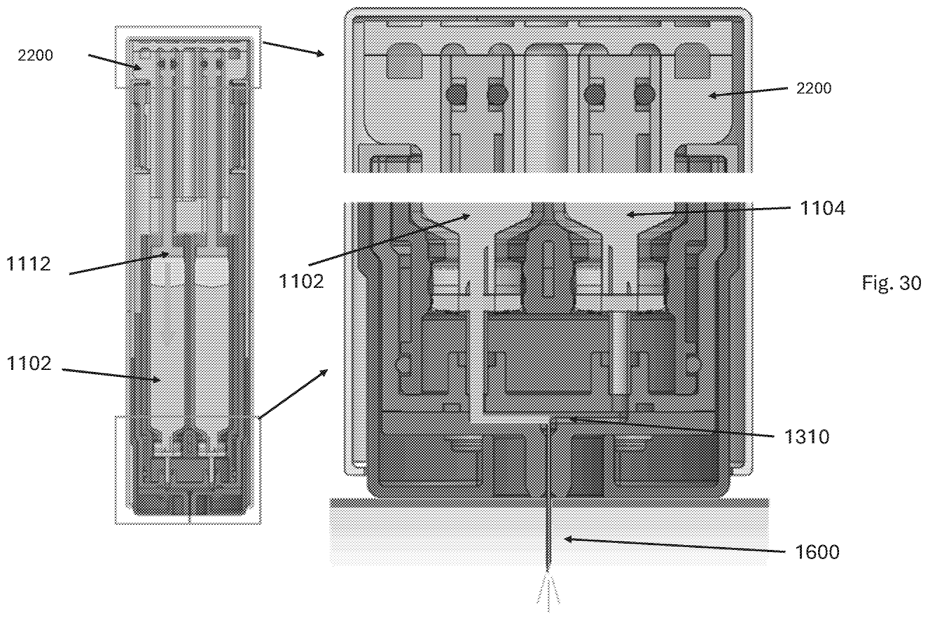

In some embodiments, immediately after the DCH Delivery Hub is activated, the first Cartridge 1102, which is pressurized by the pressurized first piston or plunger rod 1112 pushing on the first cartridge stopper, begins delivering the first drug/dose via the Flow Channel 1310 and Delivery Needle 1600. FIG. 30 depicts an exemplary embodiment, with a zoom-in view depicting dispensing of the first cartridge 1102. Although both cartridges 1102, 1104 may be pierced by needles 1302, 1304 at this stage, the contents of cartridge 1104 are not dispensed as there is no pressure applied, at this point in time, to the plunger/piston of the second cartridge to depress the plunger/piston and dispense the medicament therein since the pathway to the second container 1104 is currently closed/blocked at this stage of use.

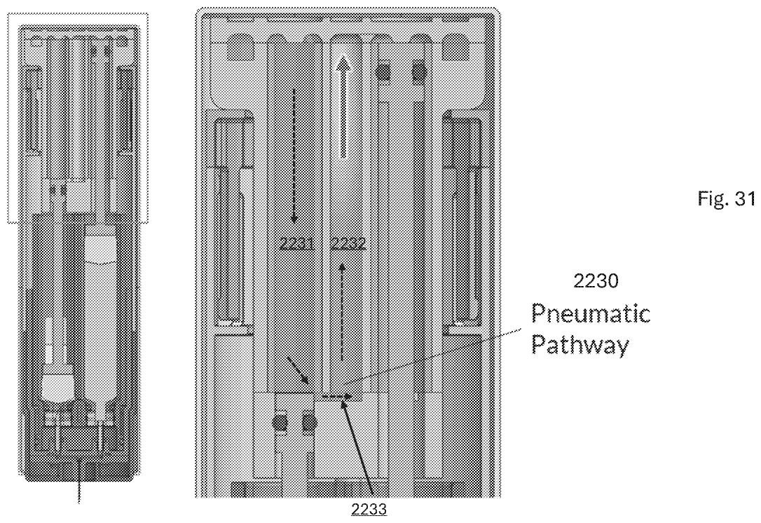

As the first dose/drug is dispensed, the first Cartridge Stopper and first Piston translate (downwardly as shown in FIG. 30). The gas overhead the first Piston expands to fill the volume within the Pneumatic Manifold 2200. Once the first plunger travels to the bottom of the first Cartridge to reach the end of deliver of the first drug/dose, the Pneumatic Pathway 2230 (from the gas cannister to the second cartridge 1104) within the Pneumatic Manifold 2200 is revealed, or opened. In the exemplary embodiment shown in FIG. 31, the pneumatic pathway extends laterally, and at the bottom, between the vertical pathways aligned with the plungers/pistons of the two drug cartridges 1102, 1104. The pneumatic pathway can be formed with right angle turns, or curved/radiused turns, as shown by the arrows in FIG. 31. Also, the pneumatic pathway 2230 can have a uniform size (e.g. diameter of the gas conduit(s)) throughout the entire pathway; alternatively, the size of the pathway can vary (e.g. smaller diameter at the lateral portion 2233 bridging the two vertical portions 2231, 2232.

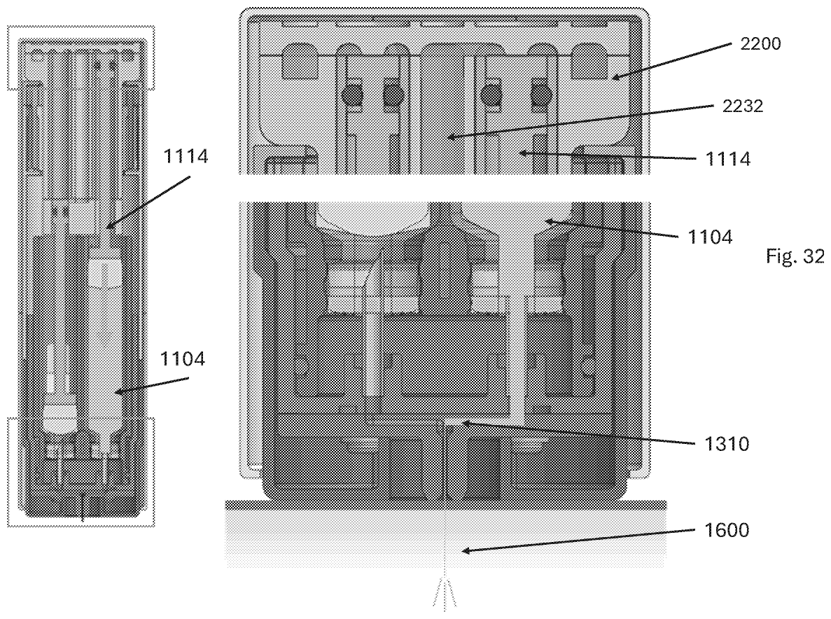

The pressurized gas within the Pneumatic Manifold is routed overhead the second piston/plunger 1114 via this Pneumatic Pathway 2230 (as well as presiding in the first container to keep the first plunger fully deployed/displaced). Next, and in some embodiments immediately thereafter, the second Cartridge 1104, which is now pressurized by the pressurized second piston/plunger 1114 pushing on the second Cartridge Stopper, begins delivering the second drug/dose via the Flow Channel 1310, as shown in FIG. 32.

As the second drug/dose is dispensed, the second Cartridge Stopper and second Piston translate (downwardly as shown in FIG. 32). The gas overhead the second Piston expands to fill the volume within the Pneumatic Manifold 2200. Once the second Cartridge 1104 nears the end of delivery, the Pneumatic Vent 2234 within the Pneumatic Manifold is revealed (e. g the plunger 1114 is displaced a sufficient distance to cease blocking the port of the vent), allowing the gas to release and for the Pneumatic Manifold 2200 to depressurize. This corresponds to the end of the Delivery State. In the exemplary embodiment, the Pneumatic Vent 2234 lies in the same horizontal plane as the lateral portion 2233 of the pneumatic pathway 2230 that bridges the two vertical portions 2231, 2232.

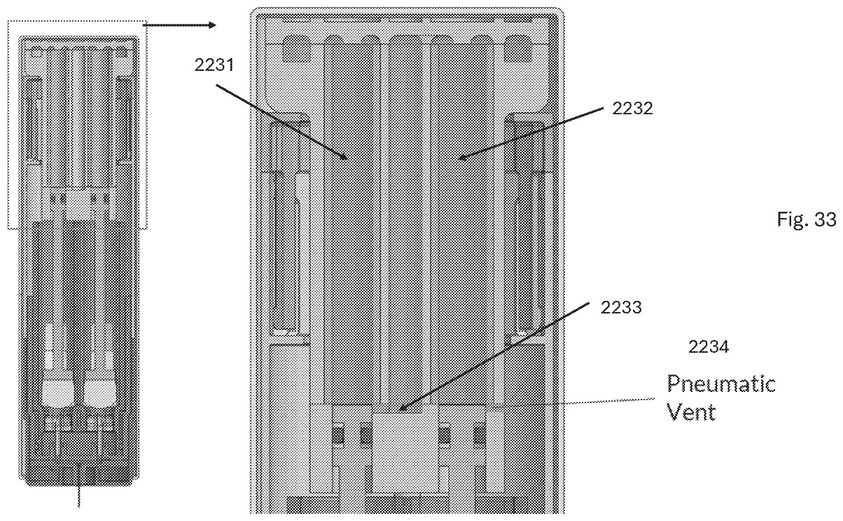

Thus, in accordance with an aspect of the disclosure, Pneumatic Pathway 2230 can include a plurality (e.g. three) vertically oriented conduits, as shown in FIG. 33. In operation, pressurized gas descends downwardly in the first vertical portion 2231, then turns in the lateral portion 2233 to reverse the gas flow direction and travel upwardly in the middle column to deliver the pressurized air (once the pathway 2233 is revealed, or opened, due to travel of first plunger 1112) above the second plunger 1114 at which point the pressurized gas turns again to flow downwardly in portion 2232 (parallel to the first pathway 2231) to depress the second plunger 1114 and dispense the second drug/dose.

### Needle Shield Lockout

In accordance with another aspect of the disclosure, as the user begins to lift the device away from the desired location, the Needle Shield Return Spring 1755, which was compressed during activation applies an opposing force on the Internal Frame 1500, which is transferred to the Needle Shield 1700.

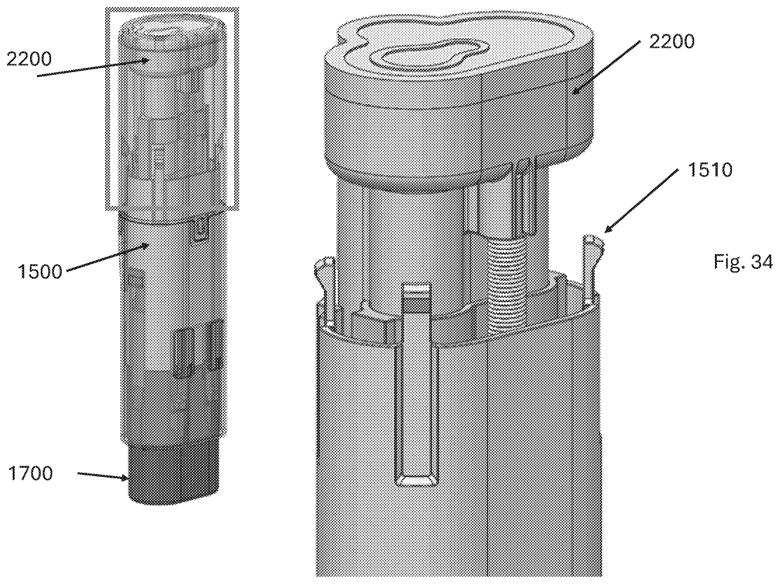

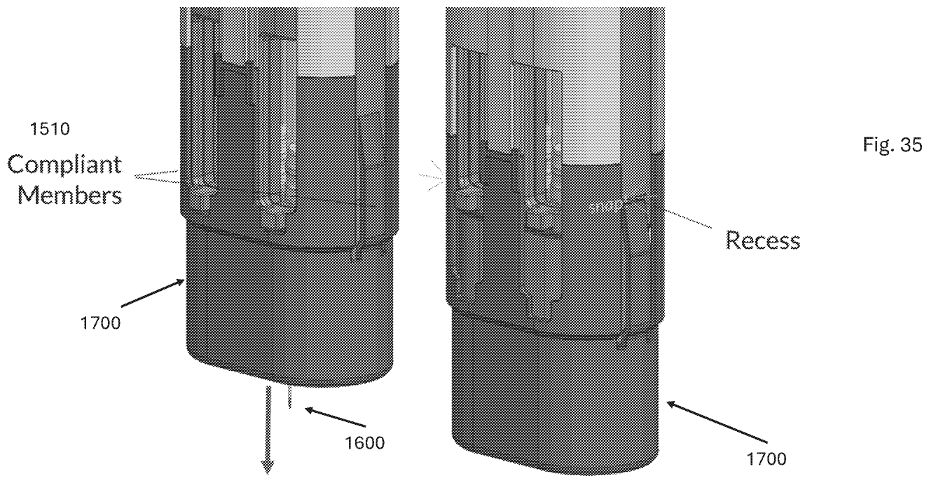

As shown in FIGS. 34-35, as the Needle Shield 1700 leaves the patient surface, it extends out of the Housing and Compliant Members 1510 snap into recesses in the Delivery Hub which were revealed as the Delivery Hub 1400 was moved during activation. The device is now in the Lockout State.

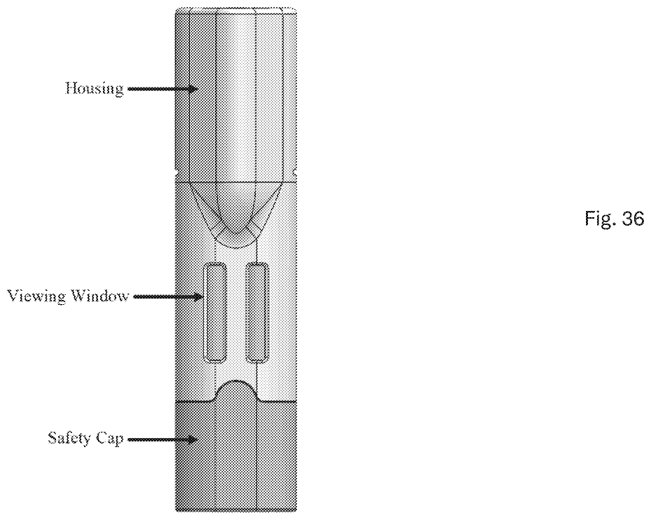

Another exemplary embodiment of a sequential delivery device in accordance with the present disclosure is shown in FIGS. 36-48. The exemplary device is a gas-powered autoinjector designed to deliver medicament(s) from two internal chambers sequentially and automatically. To use the device, a user removes the safety cap, firmly presses the needle shield onto the desired delivery site until the needle shield collapses, then waits a prescribed amount of time, during which both doses are delivered automatically. Thus, the device disclosed herein provides a two-step autoinjector, which employes a novel solution for achieving complex internal device states with limited user input.

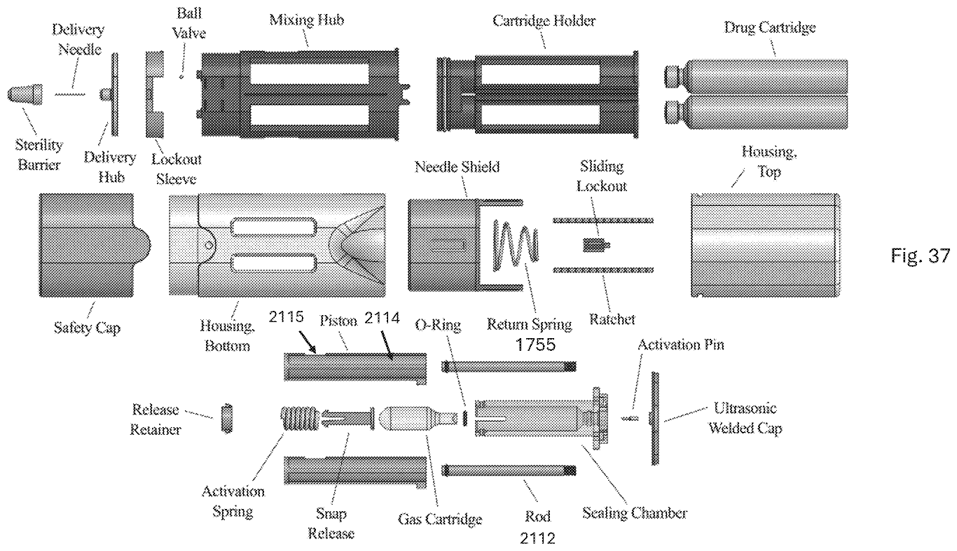

A side view of the device is shown in FIG. 36, with an exploded view of the various components of the device shown in FIG. 37.

### Nominal State

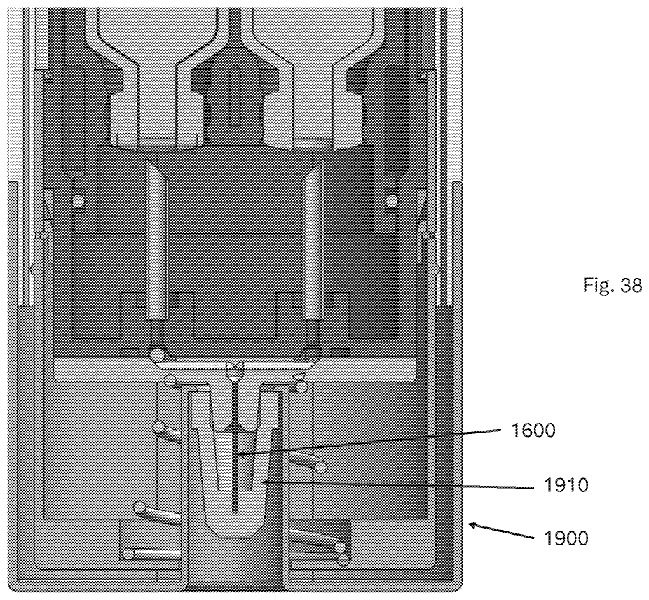

In the stored state, the device is presented as a capped autoinjector, as shown in FIG. 38. This cap 1900 captures a needle sterility barrier 1910, which maintains delivery needle 1600 sterility during storage. Users can inspect the medicament before using the device via windows (2-sided). The safety cap 1900 also prevents accidental activation of the autoinjector during device inspection and preparation by preventing user interaction with the needle shield 1700.

### Ready State

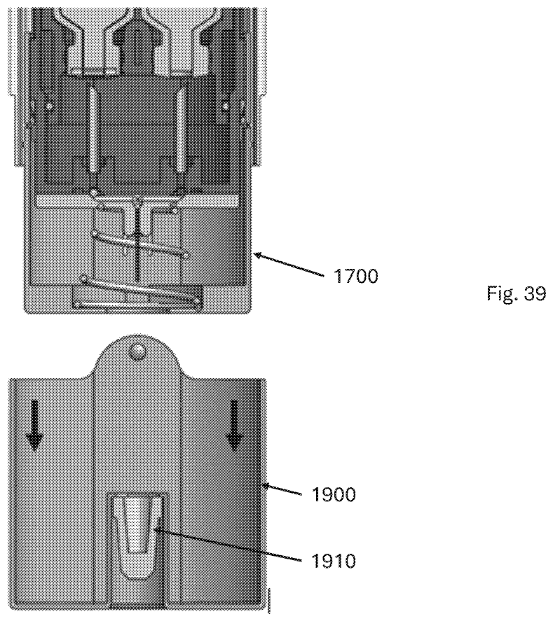

When the user is ready to perform the injection, they firmly grasp the safety cap 1900 and pull axially relative to the device body, as shown in FIG. 39. As the safety cap 1900 is removed, the needle sterility barrier 1910 is also removed from the delivery needle 1600. Once the safety cap 1900 is removed, the device is ready to deliver. The needle shield 1700 still protects user from accidental pricks by the delivery needle while they position the device.

### Activation

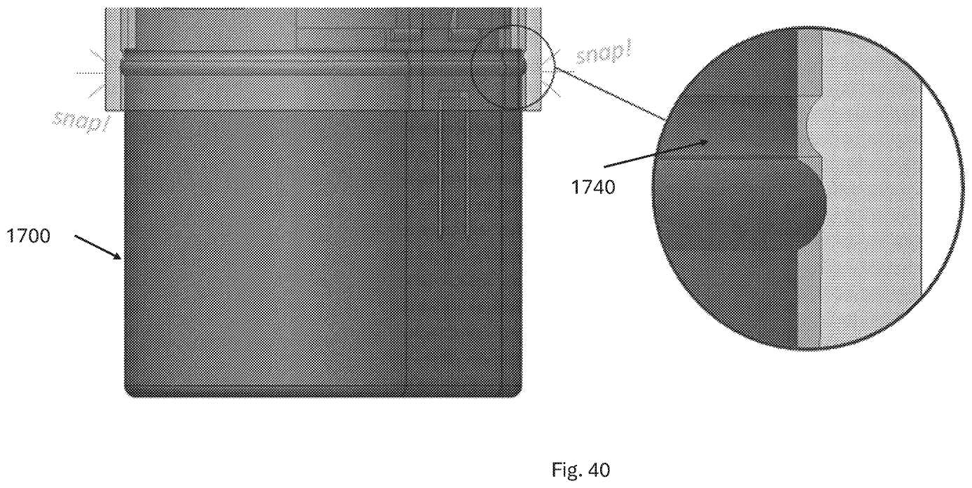

The user aligns the device with the desired delivery location and begins firmly pressing the needle shield 1700 into the delivery site. As they press, the needle shield 1700 contacts detents 1740 on the inside of the housing, resisting the user force, as shown in FIG. 40. The user must generate enough downward force to overcome the resistance force of annular detent. Once sufficient force is generated, the detent deflects around the needle shield, allowing it to continue translating upwards. This activation feature can also be included in the exemplary embodiment disclosed in connection with FIGS. 17-35.

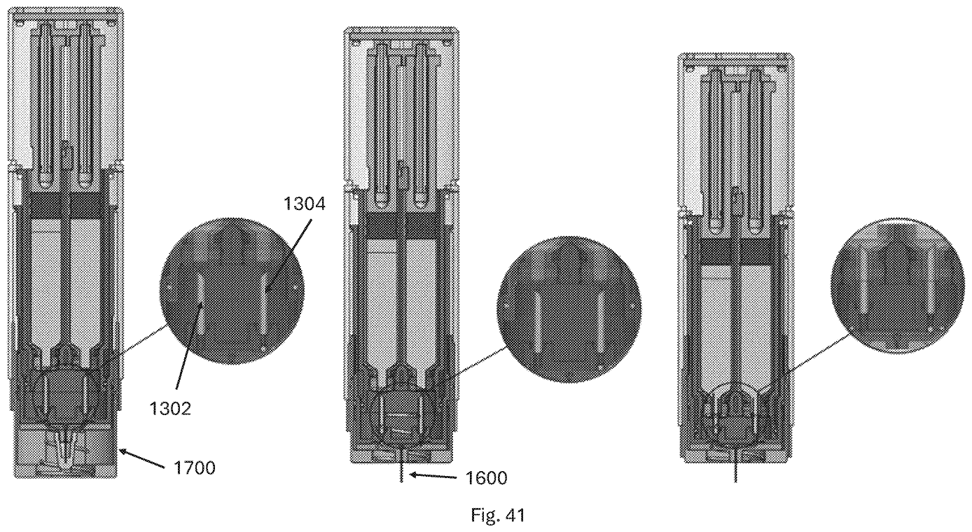

The first action once the detent 1740 has been overcome sees the delivery needle 1600 exposed from the device and inserted into the patient (as shown in the left image of FIG. 41). The needle shield then bottoms out on the needle hub of the PDC, forcing the needles 1302, 1304 into the drug cartridges (as shown in the middle image of FIG. 41). Once the needles 1302, 1304 have been fully seated, the PDC has been activated. The device has now been activated, and the next series of device states are achieved by utilizing the residual downward force provided by the user. The exact timing of each state may change relative to the other steps.

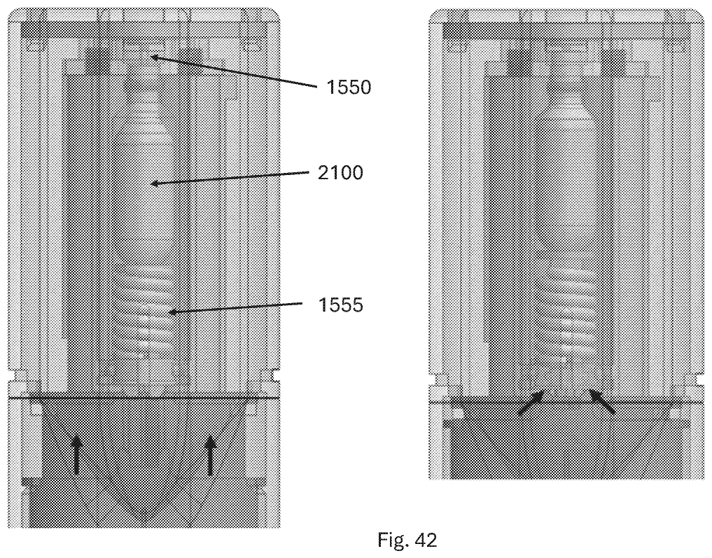

As shown in FIG. 42, at the end of travel, two upwardly projecting extensions on the PDC pinch together the snap arms of the snap release. Once the snap arms have cleared the mating flat surface, the snap release is free to move. The activation spring 1555 provides an upward force, driving the activation release and the gas cartridge 2100 upward. The gas cartridge 2100 is punctured by the activation pin 1550, allowing gas to flow into the system. An o-ring around the neck of the gas cartridge prevents gas flow back around the gas cartridge. The sealed nature of the cap (potentially ultrasonically welded (UW)) prevents gas from escaping the back end of the device. In the exemplary embodiment shown, the snap release is designed with a double snap arm release mechanism but other options are available to trigger the activation release including a cam release.

### Sequential Delivery

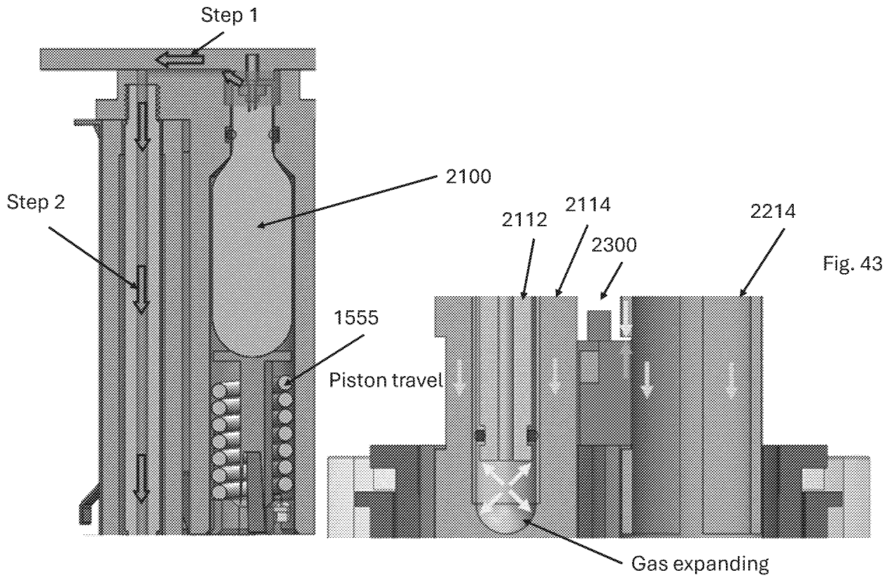

Once the gas is released from the cartridge, it moves across the top between the sealing chamber and the sealed cap (labeled Step 1) and down into the inner diameter of both rods, (labeled Step 2) as shown in FIG. 43. That is, the pressurized gas travels downward inside the inner diameter of the piston rod 2112, which itself is within an inner diameter of the piston 2114. As the pressurized gas exits the piston rod 2112, it is caught by a receiving portion (shown as a cup-shaped portion in the image on the right side of FIG. 43), which results in relative motion between the rod 2112 and the piston 2114. Once out of the bottom of the rod 2112, the gas fills the volume between the rod 2112 and the piston 2114. As the gas is continuously delivered, the volume of the gas expands, which causes the piston 2114 to be displaced downwardly (as shown by the arrows in FIG. 43) to dispense the medicament therebelow and within that cartridge.

As the pressure increases, the piston 2114 of the first dose in the first cartridge 1102 begins to translate downward, delivering the first dose. The second piston 2214 is also pressurized, and thus urged to move downwardly as shown by the arrows in FIG. 43, but is prevented from moving by the second dose lockout 2300. In some embodiments, the piston can utilize an overmolded metal tube to eliminate draft on the internal diameter of the piston, allowing for a constant seal between the rod and piston throughout travel.

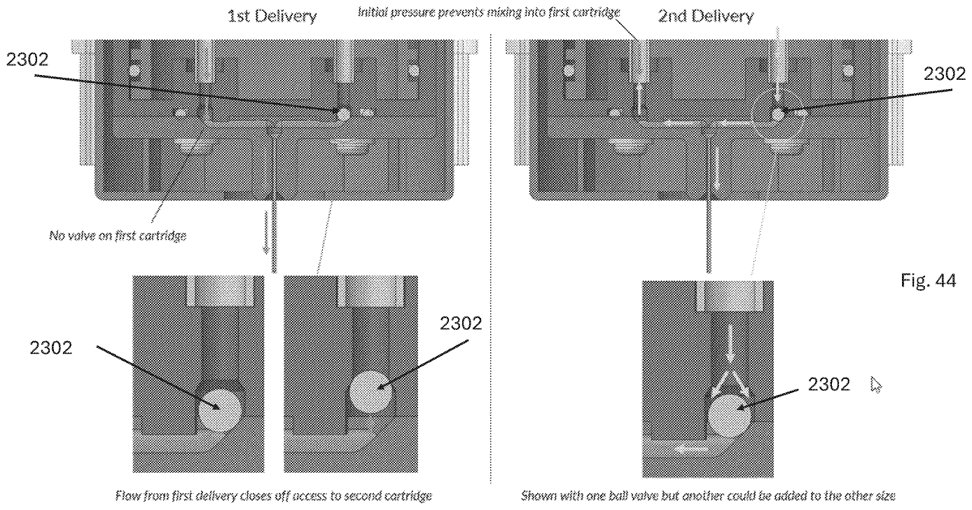

In accordance with an aspect of the disclosure, an optional feature can be incorporated into the embodiment described in connection with FIGS. 36-43. In some embodiments, mixing prevention is achieved by a one way valve (e.g. ball valve) located in the PDC above the delivery needle 1600, as shown in FIG. 44. During the dispensing of the first dose/drug from the first cartridge 1102, the ball 2302 is pushed against the wall of the Flow Channel 1310 (.e. g under the second cartridge 1104) from the pressure of the drug fluid within the channel 1310. In some embodiments, the ball can be displaced laterally and upwardly (as shown in the left side of FIG. 44), and is received within a socket that effectively closes the valve. This seals off access to the second drug cartridge 1104, limiting or prohibiting contact between the two drugs. When the first dose is complete and the second dose begins, the drug from the second cartridge 1104 can move around the ball and out the delivery needle (as shown on the right side of FIG. 44). The residual pressure from the first dose prevents mixing back into the first cartridge 1102. It should be noted that the exemplary (ball) valve feature is not a requirement of the SDA, and that sequential drug delivery can be achieved, as described above in connection with FIGS. 36-43, without employing this feature.

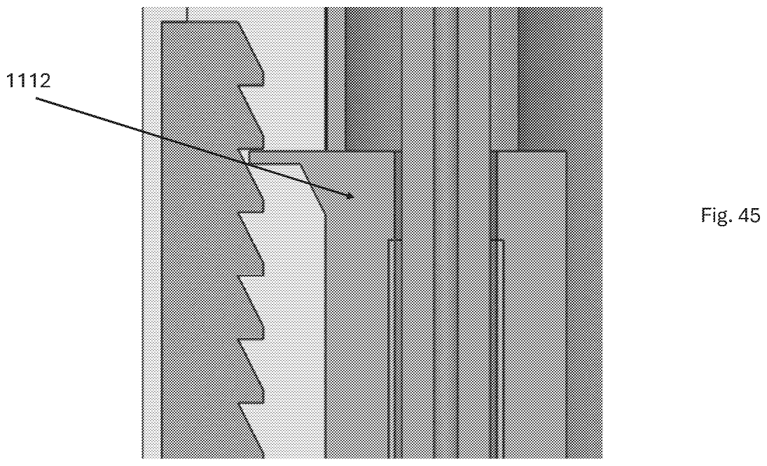

In some embodiments, as illustrated in FIG. 45, as each delivery is performed, a flexure on the back side of the piston 1112 runs along the edge of a ratchet, which can also be used to produce an audible clicking noise which indicates to the user that the device is continuing to deliver. The sound stopes once each dose has been completely delivered.

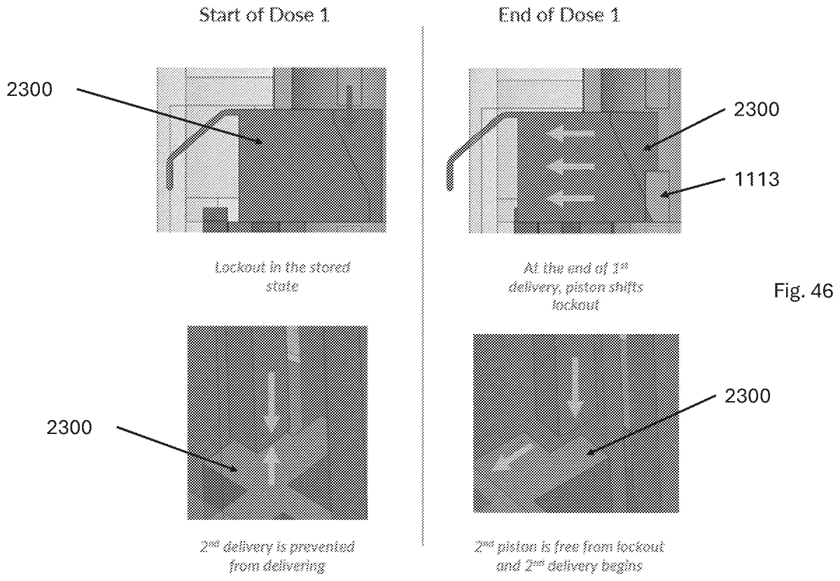

As shown in FIG. 46, at the end of travel for the first dose, an extended arm 1113 on the piston contacts an inclined plane on the sliding lockout 2300. This moves the second dose release such that the ledge preventing the second dose from being delivered is cleared from the second piston, allowing the second dose to begin. At the end of the second dose, a slit 2115 in the piston (best shown in FIG. 37) allows any extra gas to be vented.

### Needle Shield Lockout

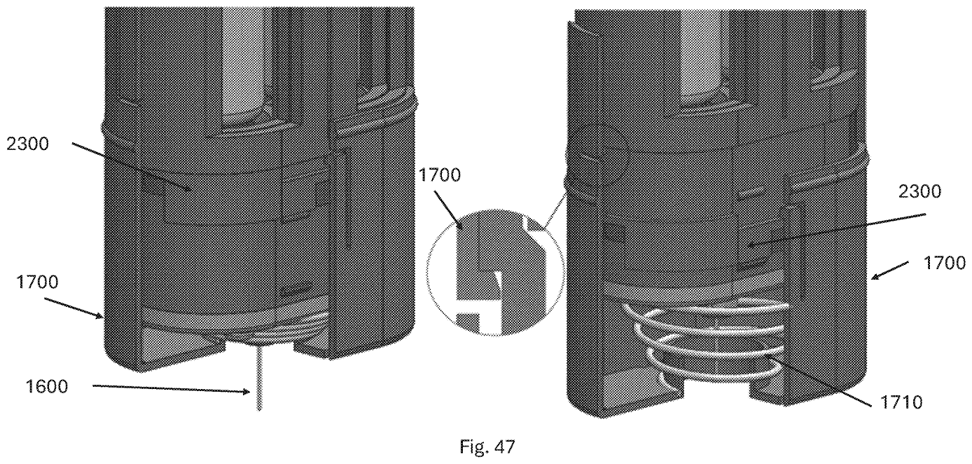

Once both doses are complete, the user pulls the device off their body. This releases the return spring 1710 which begins the lockout action. As the return spring 1710 is extended, a snap arm on the needle shield 1700 pulls down the lockout sleeve, revealing a ledge for the needle shield 1700 to lock into, as shown in FIG. 47. At the end of travel, the top snap on needle shield 1700 engages with the ledge, preventing it from depressing again. A ledge towards the bottom of the PDC prevents the needle shield from being removed entirely. As noted previously, the lock out mechanism described here can be used on any of the SDA embodiments.

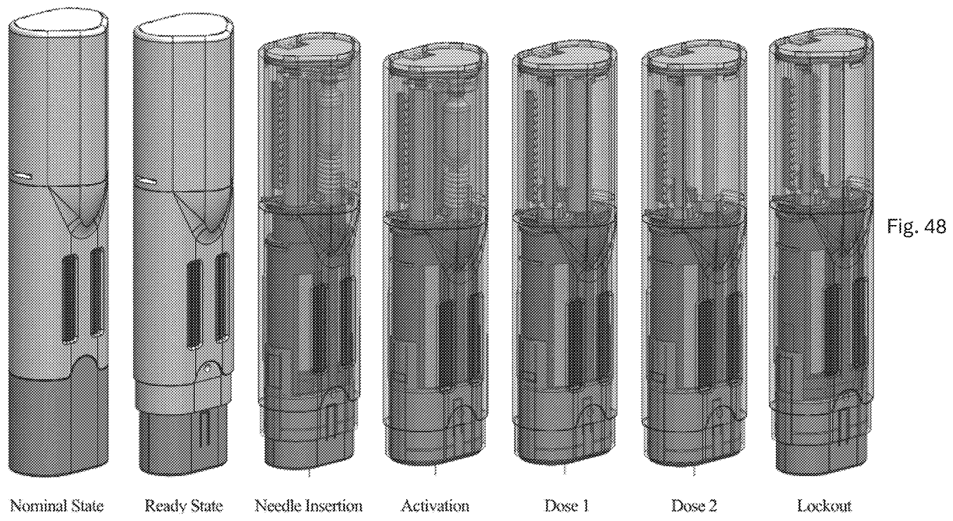

For purpose of illustration and not limitation, FIG. 48 depicts the various states of operation as described above.

As previously noted, it will be apparent to artisans of ordinary skill that although the exemplary embodiments of the present disclosure depict a two-cartridge device, additional cartridges can be included, and each can include a separate valve to permit selective opening of the valve and dispensing of the contents of its associated container. For example, a plurality of cartridges (and valves with requisite channels coupled to the cartridges) can be configured in a circular ring (similar to a gun barrel) to provide multiple stages, and substances, for dispensing.

While the disclosed subject matter is described herein in terms of certain preferred embodiments, those skilled in the art will recognize that various modifications and improvements may be made to the disclosed subject matter without departing from the scope thereof. Moreover, although individual features of one embodiment of the disclosed subject matter may be discussed herein or shown in the drawings of the one embodiment and not in other embodiments, it should be apparent that individual features of one embodiment may be combined with one or more features of another embodiment or features from a plurality of embodiments.

In addition to the specific embodiments claimed below, the disclosed subject matter is also directed to other embodiments having any other possible combination of the dependent features claimed below and those disclosed above. As such, the particular features presented in the dependent claims and disclosed above can be combined with each other in other manners within the scope of the disclosed subject matter such that the disclosed subject matter should be recognized as also specifically directed to other embodiments having any other possible combinations. Thus, the foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the method and system of the disclosed subject matter without departing from the spirit or scope of the disclosed subject matter. Thus, it is intended that the disclosed subject matter include modifications and variations that are within the scope of the appended claims and their equivalents.

1. A drug delivery system comprising:
a housing configured to hold a first container and a second container, wherein the first container contains a first medicament component and the second container contains a second medicament component;
a first seal associated with the first container;
a second seal associated with the second container;
a fluid communication assembly having a fluidic channel between the first container and the second container, the fluid communication assembly configured to be displaced from a first position to a second position within the housing thereby opening, removing or otherwise piercing the first seal and second seal to provide a fluidic pathway between the first container and the second container;
at least one stored energy source configured to dispense the first and second medicament components; and
a needle delivery assembly including a delivery needle having a first end and a second end, the first end of the delivery needle in fluid communication with the fluidic channel in the first position.

2. The system of claim 1, wherein the at least one stored energy source includes a spring engaged with a first plunger of the first container and a second spring engaged with a second plunger of the second container.

3. The system of claim 2, further comprising a plate coupled to the first and second plungers, the plate restricting displacement of at least one of the first and second plungers.

4. The system of claim 3, wherein at least one of the first plunger and second plunger includes a structural feature configured to displace the plate, thereby permitting displacement of the other plunger.

5. The system of claim 1, wherein the at least one stored energy source includes at least one pressurized gas chamber, the at least one pressurized gas chamber in communication with the first container via a first pathway and the second container via a second pathway.

6. The system of claim 5, further comprising a valve disposed between the at least one gas chamber and at least one of the first and/or second containers, the valve selectively opening flow of pressurized gas to one of the first and/or second containers.

7. The system of claim 6, further comprising a third pathway in communication with the at least one pressurized gas chamber and at least one of the first pathway and second pathway.

8. The system of claim 5, the at least one pressurized gas displaces a plunger in the first container a predetermined distance to open the second pathway to the second container.

9. The system of claim 5, wherein the at least one gas chamber includes a first gas chamber in fluid communication with the first container and a second gas chamber in fluid communication with the second container.

10. The system of claim 5, wherein the first gas chamber displaces a first plunger in the first container a predetermined distance to open the second gas chamber, thereby displacing a second plunger in the second container.

11. The system of claim 10, wherein the predetermined distance is detected by a sensor.

12. A drug delivery system comprising:
a housing configured to hold a first container and a second container, wherein the first container contains a first medicament component and the second container contains a second medicament component;
a first seal associated with the first container;
a second seal associated with the second container;
a fluid communication assembly having a fluidic channel between the first container and the second container, the fluid communication assembly configured to be displaced from a first position to a second position within the housing thereby opening, removing or otherwise piercing the first seal and second seal to provide a fluidic pathway between the first container and the second container;
a pressurized gas chamber at least partially disposed in the housing and in fluid communication with the first container and the second container;
an activation mechanism configured to open or otherwise pierce the pressurized gas chamber;
at least one valve configured to release a portion of pressurized gas that facilitates the dispensing of the first and second medicaments components; and
a needle delivery assembly configured to be in fluid communication with the first and second containers during a delivery phase.

13. The system of claim 12, wherein the pressurized gas flows through a first pressurized gas pathway to displace a plunger in the first container a predetermined distance, thereby opening a second pressurized gas pathway to the second container.

14. The system of claim 13, wherein pressurized gas remains within the first pressurized gas pathway while the pressurized gas flows to the second pressurized pathway.

15. The system of claim 12, wherein the first medicament and second medicament are dispensed sequentially and automatically.

16. The system of claim 12, wherein the pressurized gas chamber is disposed above the first container and a second container.

17. A drug delivery system comprising:
a housing configured to hold a first container and a second container, wherein the first container contains a first medicament component and the second container contains a second medicament component;
a first seal associated with the first container;
a second seal associated with the second container;
a fluid communication assembly having a fluidic channel between the first container and the second container, the fluid communication assembly configured to be displaced from a first position to a second position within the housing thereby opening, removing or otherwise piercing the first seal and second seal to provide a fluidic pathway between the first container and the second container;
a pressurized gas chamber at least partially disposed in the housing and in fluid communication with the first container and the second container;
an activation mechanism configured to open or otherwise pierce the pressurized gas chamber;
at least one valve configured to release a portion of pressurized gas that facilitates the dispensing of the first and second medicaments components; and
a needle delivery assembly configured to be in fluid communication with the first and second containers during a delivery phase; and
a valve disposed within the fluidic channel between the first container and the second container.

18. The system of claim 17, wherein the valve closes the fluidic channel to the second container while the medicament from the first container is dispensed.

19. The system of claim 18, wherein the valve is a ball valve, with the first medicament component displacing the ball valve a first direction to close the fluid channel of the second container.

20. The system of claim 19, wherein the second medicament component displaces the ball valve a second direction to close the fluid channel of the first container.
